(19) United States
(12) Patent Application Publication
(10) Pub. No.: US 2025/0229028 A1
(43) Pub. Date: Jul. 17, 2025

(54) DISPENSING DEVICE INCORPORATING DIAPHRAGM

(71) Applicant: AKTIVAX, INC., Broomfield, CO (US)

(72) Inventor: Amir Genosar, Broomfield, CO (US)

(21) Appl. No.: 19/115,153

(22) PCT Filed: Mar. 13, 2023
(86) PCT No.: PCT/US2023/064265

§ 371 Date: Mar. 25, 2025

Related U.S. Application Data
(60) Provisional application No. 63/319,139, filed on Mar. 11, 2022.

(51) Int. Cl. A61M 5/30; A61J 1/14; A61J 1/20

(52) U.S. Cl. CPC A61M 5/3007; A61J 1/1406; A61J 1/201

(57) ABSTRACT

A dispensing device for dispensing a beneficial agent configured to transition between an inactive state and an active state. The dispensing device can include a body, a pressure chamber, a primary container, and/or a diaphragm. The pressure chamber and diaphragm may both be disposed within the body, and the primary container may be disposed within the pressure chamber and engaged with the diaphragm. The primary container may include a distal end with a septum or pierceable fitment disposed on a distal side of the diaphragm. The dispensing device may be configured to transition between an inactive state and an active state when a pressurized fluid is provided to the pressure chamber by moving the primary container distally against the diaphragm to cause the diaphragm to deform, including stretching a section of the diaphragm in a distal direction.

### CROSS-REFERENCE TO RELATED APPLICATIONS

This application claims benefit of priority to U.S. Provisional Application No. 63/319,139 filed on Mar. 11, 2022, and incorporates that application by reference for all it contains. In the event of a conflict in the definition or use of any term between the priority application and the present application, the definition or use in the present application controls.

### FIELD

The present disclosure pertains to the field of parenteral drug delivery of a beneficial agent and, more particularly, to prefilled injection devices.

### BACKGROUND

Pressure-driven drug administration devices cover a wide array of applications, including virtually every form of enteral and parenteral drug delivery. In particular, auto-injectors are used for the clinical parenteral administration of beneficial agents to subjects. Jet injectors are a particular form of auto-injector, and they can deliver a beneficial agent to intradermal, subcutaneous, and intramuscular sites through a narrow, high-pressure stream that penetrates the tissue. Today's jet injectors used for human subjects may be single-use, disposable devices that avoid the risk of subject-to-subject contamination. Because they lack needles, jet injectors require minimal training for proper use and eliminate needle stick wounds commonly seen in subjects due to improper administration using needle-based delivery devices. The ease of use promotes rapid delivery of biological agents, which is beneficial in emergency settings and when situations demand administration to large numbers of individuals. Other examples of potential use include veterinary applications for the delivery of medical reagents for animal care, promoting safety for the animal and the human caretaker. When in a single-handed auto-injector configuration, the benefits of a jet injector increase. Notably, there is a further decrease in the complexity of administration, allowing the delivery of agents with fewer steps than traditional injections.

Jet injectors rely on an applied pressure to eject the beneficial agent at high velocity through an orifice to cross the epidermal and dermal tissues. This force frequently originates from a mechanically induced pressure supplied by an external source of carbon dioxide or nitrogen, a loaded spring, or an electric power supply, each of which drives a piston assembly. The piston, in turn, rapidly compresses the chamber containing the beneficial agent resulting in increased chamber pressure and the expulsion of the solution.

### SUMMARY OF THE DISCLOSURE

The present disclosure describes a dispensing device capable of dispensing a beneficial agent and configured to transition between an inactive state, wherein a beneficial agent is stored in a storage volume, and an active state, wherein a beneficial agent is being dispensed. Forms of the dispensing device may include an auto-injector capable of administering beneficial agents through needle injection or jet injection. The dispensing device may include a primary container, an activator, a pressure chamber, a diaphragm, a proximal end, and a distal end which may include a distal port. The activator, which may be a button, may be defined by a set of rigid walls and located at a proximal end of the dispensing device, projecting out of the body of the auto-injector. The activator may be disposed at a proximal portion of the pressure chamber. The volume of the pressure chamber may be defined by a sidewall and a flexible diaphragm, and a central barrel may be disposed therewithin.

The dispensing device incorporates a primary container, which may be situated within the pressure chamber and engaged with the diaphragm. The primary container includes a storage volume for storing a beneficial agent and may be configured to deplete the storage volume through a distal end when a pressurized fluid is provided to the pressure chamber. In one variation, the primary container may be a flexible cartridge configured to collapse in the active state. In another variation, the primary container may be a rigid cartridge defined by rigid walls, a plunger configured to seal the storage volume against the volume of the pressure chamber, and a distal container port which may include a septum. When a pressurized fluid is provided to the pressure chamber, the force of the pressurized fluid may cause distal movement of the primary container and distal stretching of a section of the diaphragm. The distal movement may bring the primary container into fluid communication with an external environment, including fluid communication through the distal port. In variations using a flexible container, the pressurized fluid may pressurize the flexible container causing it to collapse and dispense the beneficial agent. In variations using a rigid container, the pressurized fluid may cause distal movement of the plunger to dispense the beneficial agent.

The dispensing device may incorporate a piercing element which may include a fluid passageway. Fluid communication between the storage volume and the external environment in the active state may be established when the piercing element pierces the distal port of the primary container. The pressure differential between the pressure chamber and the ambient environment results in continued force against the primary container and the expulsion of the beneficial agent contained within the storage volume through the distal port. The beneficial agent may be attached externally or stored internally within a glass or polymer chamber of the primary container that may or may not be replaceable. The description contained herein is not intended to limit the disclosure, and each embodiment may be used independently of, or in combination with, features of other embodiments.

### BRIEF DESCRIPTION OF THE DRAWINGS

FIG. 1a illustrates a dispensing device including a diaphragm and a sealed second region.

FIG. 1b illustrates a cross-section view of a jet injector, including a diaphragm and a sealed second region in an inactive state pre-activation.

FIG. 1c illustrates a cross-section view of a jet injector, including a diaphragm and a sealed second region active state.

FIG. 2 illustrates a section view of a jet injector, including a diaphragm, wherein the piercing region of the diaphragm is sealed.

FIG. 3 illustrates a section view of a jet injector, including a diaphragm wherein the sealing of the diaphragm to the package is enhanced by a torus spring.

FIG. 4 illustrates a section view of a jet injector, including a diaphragm with a retraction spring biasing the package toward the first position.

FIG. 5 illustrates a section view of a jet injector, including a diaphragm wherein the diaphragm comprises a bleed hole.

FIG. 6a illustrates a section view of a jet injector, including a diaphragm wherein the second region comprises a support wall in a pre-activation inactive state.

FIG. 6b illustrates a section view of a jet injector, including a diaphragm wherein the second region comprises a support wall in an active state.

FIG. 7 illustrates a section view of a jet injector, including a diaphragm wherein a regulated bleed pathway is formed between the diaphragm and the support wall.

FIG. 8 illustrates a section view of a jet injector, including a diaphragm wherein the second region comprises a vent hole.

FIG. 9 illustrates a section view of a jet injector, including a flexible, collapsible package and a retraction spring biasing the package toward the first position.

FIG. 10 illustrates a section view of a jet injector, including a diaphragm wherein the diaphragm comprises a stress relief fold in the diaphragm structure.

FIG. 11a illustrates a section view of an auto-injector, including a diaphragm and an injection needle in an undeployed inactive state.

FIG. 11b illustrates a section view of an auto-injector, including a diaphragm and an injection needle in deployed active state.

FIG. 12 illustrates a section view of an auto-injector, including a diaphragm and an injection needle wherein the diaphragm seals against the needle hub.

FIG. 13 illustrates a section view of an auto-injector, including a diaphragm and an injection needle wherein the diaphragm seals against the needle sheath.

### DETAILED DESCRIPTION

The disclosure relates to a dispensing device for dispensing a beneficial agent that is configured to transition between an inactive state and an active state. The dispensing device can include a body, a pressure chamber, a primary container, and/or a diaphragm. The pressure chamber and diaphragm may both be disposed within the body, and the primary container may be disposed within the pressure chamber and engaged with the diaphragm. The primary container may include a distal end with a septum or pierceable fitment disposed on a distal side of the diaphragm. The dispensing device may be configured to transition between an inactive state and an active state when a pressurized fluid is provided to the pressure chamber by moving the primary container relative to (e.g., distally against) the diaphragm to cause the diaphragm to deform, including stretching a section of the diaphragm in a distal direction.

Forms of the dispensing device may include beneficial agent dispensers for intranasal, ocular, otic, oral, rectal, topical, transdermal, intravenous, subcutaneous, intramuscular, or any other enteral or parental form of beneficial agent administration. The dispensing device may be an auto-injector capable of administering beneficial agents through needle injection or jet injection, and such administration will typically be parental administration, such as subcutaneous, intramuscular, or intravenous administration. Some variations may be configured for direct-to-organ administration, such as intracardiac, intrapleural, epidural, or knee injections. The dispensing device may incorporate an administration element at the distal end, which could include any of a needle, a jet injector nozzle, a cannula, a tapered fitting, a medical device connector, or any equivalent element through which the beneficial agent may be depleted. The dispensing device may include a primary container, activator, pressure chamber, diaphragm, and/or a distal end which may include a distal port. The activator, which may be a button, can have rigid walls and may be located at a proximal end of the dispensing device, projecting out of the body. The activator may protrude into a proximal portion of the pressure chamber. The volume of the pressure chamber may be defined by a primary wall and a flexible diaphragm and may form a hollow cylindrical volume within the body.

The dispensing device may incorporate a primary container, which may be situated within the pressure chamber and engaged with the diaphragm. The diaphragm may optionally include a receptacle with which the primary container is engaged, and the primary container may optionally include a fitment that may be engaged with the receptacle. The primary container may include a storage volume for storing a beneficial agent and may be configured to deplete the storage volume through a distal end when a pressurized fluid is provided to the pressure chamber. In one variation, the primary container may be a flexible cartridge configured to collapse in the active state when a pressurized fluid is provided into the pressure chamber. In another variation, the primary container may be a rigid cartridge which can include rigid walls, a plunger optionally including a plunger rod, and/or a stopper which may be configured to bound the storage volume against the volume of the pressure chamber and/or an interstitial volume between the plunger and stopper. A rigid cartridge may include a distal end with a septum pierceable by a piercing end of an administration device.

When a pressurized fluid is provided to the pressure chamber, the force of the pressurized fluid may cause distal movement of the primary container and distal stretching of a section of the diaphragm. The distal movement may bring the storage volume of the primary container into fluid communication with an external environment, which may include fluid communication through the administration device and/or the distal port. The distal stretching of the section of the diaphragm may allow for movement of the primary container within the dispensing device while maintaining an elevated pressure in the pressure chamber (e.g., at least during dispensing of the beneficial agent from the primary container). In variations with a flexible cartridge, the pressurized fluid may apply pressure to the flexible container causing it to collapse and dispense the beneficial agent. In variations using a rigid cartridge, the pressurized fluid may cause distal movement of the plunger, plunger rod, and/or stopper to deplete the storage volume and dispense the beneficial agent. Dispensing the beneficial agent may include a flow of a fluid beneficial agent through the administration element and/or the distal port.

The administration element may incorporate a piercing end and/or a fluid passageway. Fluid communication between the storage volume and the external environment in the active state may be established when the piercing element pierces the distal end of the primary container. The pressure differential between the pressure chamber and the ambient environment results in continued force against the primary container and the expulsion of the beneficial agent contained within the storage volume through the distal port. The beneficial agent may be attached externally or stored internally within a glass or polymer chamber of the primary container that may or may not be replaceable.

The distal end of the dispensing device may further include an administration element at least partially disposed on the distal side of the diaphragm in the inactive state. The administration element may be configured to penetrate the primary container responsive to the distal moving of the primary container (e.g., through the distal stretching of the portion of the diaphragm). The primary container may optionally be at least partially sealed by a septum, which may be made of an elastomeric material able to close when an administration element is withdrawn from the primary container after a dwell time. The administration element may optionally be configured to penetrate the primary container through the diaphragm and/or a septum responsive to the distal moving of the primary container.

The dispensing device may optionally further include a pressurized fluid source, and the pressurized fluid source may be adapted to provide the pressurized fluid to the pressure chamber. The pressurized fluid source may include a pressurized gas canister, a gas-generating chemical reaction, a pump, or any equivalent structure. The pressurized fluid may be communicated into the pressure chamber through an entry port, which may be situated in or near a pressurized fluid source initiator. The pressurized fluid source may be arranged to move distally when an activator is activated. An activator may include a button, lever, twist knob, or another mechanical actuator. Distal movement of the pressurized fluid source may cause it to interact with a pressurized fluid source initiator such that the pressurized fluid is provided by the pressurized fluid source.

The receptacle may be compressed against the primary container when a pressurized fluid is provided to the pressure chamber, and the receptacle may optionally compress against a fitment included in the primary container. Such compression of the receptacle may be at least in part in response to the distal stretching of the portion of the diaphragm. That is, as the diaphragm stretches distally, the receptacle may be constricted to provide the compressive force against the primary container to assist in the sealing of the primary container to the receptacle such that the beneficial agent is expelled through the administration element. Such a fitment may be disposed substantially within the primary container and may optionally comprise a formed rigid shape. A retaining element may optionally be disposed relative to the receptacle and may be adapted to retain the receptacle against the primary container. The retaining element may optionally be adapted to maintain a seal between the receptacle and the primary container.

The distal moving of the primary container may be responsive to the pressurized fluid source providing the pressurized fluid to the pressure chamber. The primary container may further include a plunger which may be positioned proximal to the storage volume. The plunger may include a plunger rod extending distally from the plunger. The plunger rod may extend into an interior cavity defined within a stopper, and the plunger and/or stopper may define a proximal boundary of the storage volume in the inactive state of the dispensing device. The transition of the dispensing device between the inactive state and the active state may optionally include distal movement of the plunger, plunger rod, and/or stopper within the primary container, and such distal movement may be responsive to the penetration of the storage volume of the primary container by an optional piercing end of the administration element. In the active state of the dispensing device, the plunger, plunger rod, and/or stopper may move distally within the primary container to deplete the storage volume, and this may expel the beneficial agent through the administration element and/or a distal port which may be located at the distal end of the dispensing device.

In some variations of the dispensing device, the primary container may include a flexible cartridge. The transition of the dispensing device between the active state and the inactive state may include flexing of the flexible cartridge, which may be responsive to providing a pressurized fluid into the pressure chamber and/or penetration of the storage volume of the flexible cartridge by the administration element. In the active state of the dispensing device, the flexible cartridge may flex, and flexing of the flexible cartridge may deplete the storage volume and may then expel the beneficial agent through the administration element and/or the distal end.

The administration element of any design of the dispensing device described in this application may include a needle, a jet injector nozzle, a cannula, a tapered fitting, and/or a medical device connector. The administration element may optionally be configured to provide fluid communication between the storage volume and an external environment in the active state.

The diaphragm may optionally include an administration element receptacle that may movably accommodate the administration element, and the administration element receptacle may be located distal to the primary container at the distal end of the dispensing device. A portion of the diaphragm may optionally be adapted to deform responsive to the distal moving of the primary container. The deformation may be such that the administration element and/or a piercing end thereof penetrates the primary container.

The dispensing device may optionally further include a barrier, which may be configured to seal the distal end against an external environment in the inactive state. Such a barrier may be configured to provide an aseptic seal that prevents the intrusion of pathogens, pyrogens, or other environmental contaminants into the dispensing device at the distal end. The administration element may optionally be configured to penetrate the barrier in the active state, and/or the barrier may be configured to be partially or wholly removed prior to activation of the dispensing device.

The diaphragm may provide a fluid-tight seal and may aseptically seal the administration element. The administration element may be sealed by a sheath, which may optionally be a component of the diaphragm. The dispensing device may further include an administration element hub, and the diaphragm may optionally seal against the distal end of the dispensing device via the administration element hub in the inactive state. The primary container may optionally be disposed on a proximal side of the diaphragm.

In some designs of the dispensing device, a fluid passage may optionally be provided. The arrangement may be such that a fluid flow of the pressurized fluid may flow through the fluid passage from the pressure chamber when a pressurized fluid is provided to the pressure chamber. The fluid passage may optionally provide a fluid flow through the diaphragm. The primary container may be configured to retract proximally when the pressurized fluid is expelled from the pressure chamber through the fluid passage. A dwell time during which the dispensing device may remain in the active state may be defined and may optionally be defined by a retraction force acting on the primary container and a flow rate of the fluid flow through the fluid passage. The storage volume of the primary container may optionally be in fluid communication with an external environment external to the dispensing device during the substantially the entire dwell time. A distal chamber may optionally be provided and may be disposed on the distal side of the diaphragm within the dispensing device. The volume of the distal chamber may be defined by one or more distal chamber walls, which may include a distal chamber sidewall and/or a distal end wall. Any feature located at the distal end of the dispensing device may optionally be disposed partially or wholly within the distal chamber. A proximal bound of the distal chamber may be provided by the diaphragm. The distal chamber may be adapted such that the optional fluid flow from the pressure chamber through the fluid passage may be directed into the distal chamber in the active state.

The arrangement of the dispensing device may be such that the primary container can retract proximally when the pressure of the pressurized fluid within the pressure chamber is lower than a threshold pressure. The dispensing device may include a biasing element such as a spring, and the biasing element may provide a force on the primary container biasing it in the proximal direction. The diaphragm may optionally be adapted to bias the primary container to retract proximally, such as by forming a diaphragm from an elastomeric material with a defined elastic modulus. The distal chamber may optionally be sealed against fluid communication and may contain a sealed fluid under pressure, providing a proximal bias to the primary container. The primary container may be configured to retract proximally when the pressure of the pressurized fluid is lower than a threshold pressure. The threshold pressure and/or total biasing force may be provided, defined, and/or modulated by the pressure of the pressurized fluid within the distal chamber, a sealed fluid within the distal chamber, a biasing element which may be disposed at the distal end of the dispensing device, and/or the diaphragm in any combination.

In some designs of the dispensing device, stretching of the diaphragm may cause a portion of the diaphragm to move to engage with the distal end of the dispensing device. A distal portion of the primary container may be sealed against a sealing portion of the diaphragm when the dispensing device is in the inactive state. A seal-assist biasing element may be included, and may be configured to bias a distal portion of the primary container against a sealing portion of the diaphragm.

The dispensing device may optionally include a distal vent hole, which may be configured to relieve pressure in the distal chamber and/or the distal end. This pressure relief may be responsive to the stretching of the diaphragm.

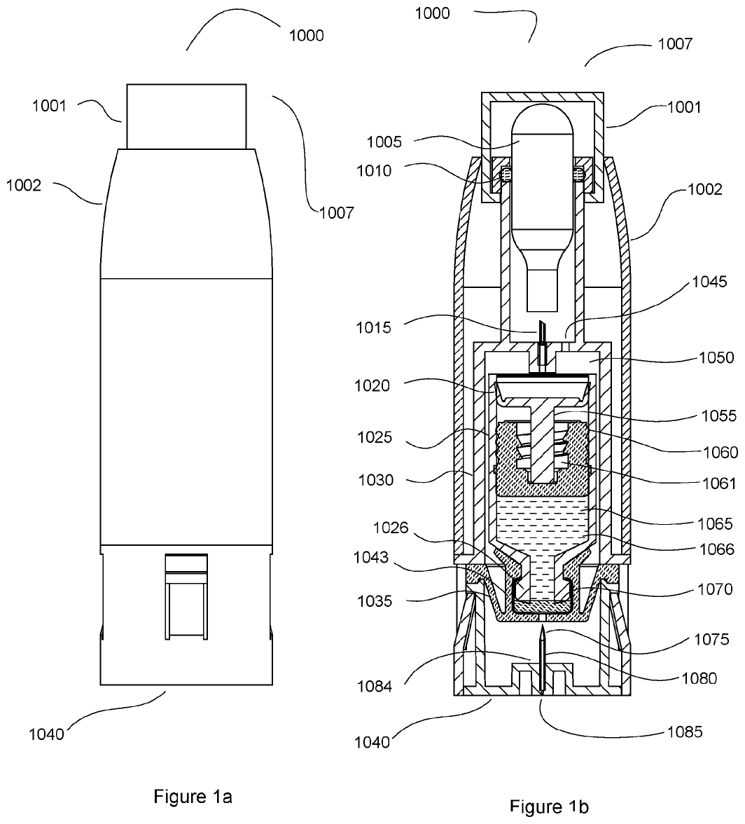

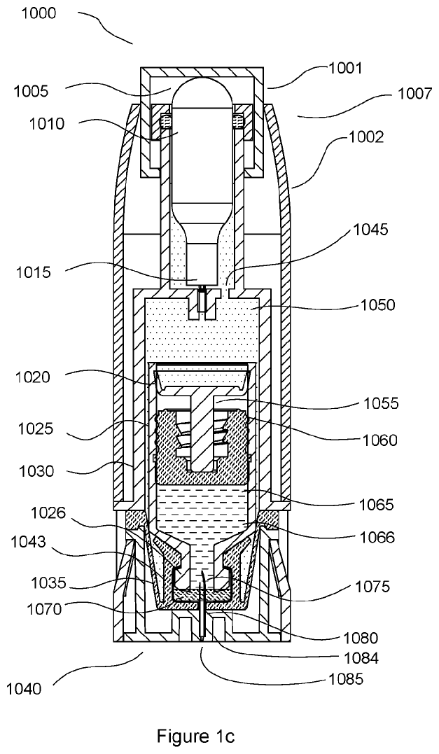

FIGS. 1a-1c illustrates a jet injector version of the dispensing device 1000 which includes an activator 1001 at the proximal end 1007 of a body 1002 housing the internal features of the dispensing device 1000. FIG. 1a depicts the external features of the dispensing device 1000 including the activator 1001, optionally comprising a button, and the body 1002. FIG. 1b illustrates a cross-sectional view of the dispensing device 1000 in an initial pre-activation state. A pressurized fluid source 1005 may be disposed relative to the activator 1001 such that application of a downward force on the activator 1001 may cause the pressurized fluid source 1005 to interact with a fluid source initiator 1015 and cause a pressurized fluid to be provided into the proximal end 1007. The movement of the activator 1001 may be facilitated by a proximal retaining element 1010, which may include a gasket, ball bearings, wheels, and/or gears and may provide high or low resistance to distal translation of the pressurized fluid source 1005. An entry passage 1045 may provide fluid communication into the pressure chamber 1050. In the initial state, a plunger 1020 may be disposed proximally within a primary container 1025 and may include a plunger rod 1055 extending distally into an interior cavity 1061 of a stopper 1060. The primary container 1025 may comprise a storage volume 1066 disposed distally relative to the stopper 1060 and storing a beneficial agent 1065. The pressure chamber 1050 may include a sidewall 1030, optionally comprising a rigid material and situated within the body 1002.

As shown in FIGS. 1a&1b, the primary container 1025 may be situated within the pressure chamber 1050 and engaged with the diaphragm 1035. The diaphragm 1035 may include a receptacle 1043 with which the primary container 1025 is engaged, and the primary container 1025 may optionally include a fitment 1026 engaged with the receptacle 1043. The primary container 1025 may include a storage volume 1066 for storing a beneficial agent 1065 and may be configured to deplete the beneficial agent 1065 through a distal port 1085 located at the distal end 1040 when a pressurized fluid is provided to the pressure chamber 1050. In one variation, the primary container 1025 may be a flexible cartridge configured to collapse in the active state when a pressurized fluid is provided into the pressure chamber 1050. In another variation, the primary container 1025 may be a rigid cartridge which can include a rigid sidewall, a plunger 1020, optionally including a plunger rod 1055, and/or a stopper 1060 which may be configured to bound the storage volume 1066 against the volume of the pressure chamber 1050 and/or an interstitial volume between the plunger 1020 and stopper 1060. A rigid cartridge may include a distal end with a septum 1070 pierceable by a puncturing end 1075 of an administration element 1080.

The distal container end of the primary container 1025 may comprise a septum 1070, which may be formed of a resealable elastomeric material. A distal end of the primary container 1025 may attach to a diaphragm 1035. The diaphragm 1035 may comprise a flexible elastomeric material and may be configured such that it will distally extend in response to the pressurization of the pressure chamber 1050 upon activation. An administration element 1080 may be situated distally relative to the distal end of the primary container 1025, held in place by an administration element hub 1084, and may comprise a puncturing end 1075. The configuration is such that the primary container 1025 may translate distally upon pressurization of the pressure chamber 1050, extending the diaphragm 1035 and causing the septum 1070 to impinge upon the puncturing end 1075 of the administration element 1080 to provide fluid communication between the storage volume 1066 and an external environment through the distal port 1085. FIG. 1c depicts the jet injector in the activated configuration. A downward force exerted on the activator 1001 translates a pressurized fluid source 1005 distally in such a manner that it may interact with a fluid source initiator 1015 causing the release of gas that may pass through an entry passage 1045 and into the pressure chamber 1050. Accumulation of a pressurized fluid within the pressure chamber 1050 may distribute pressure across a surface of the plunger 1020 and induce distal movement of the primary container 1025 within the pressure chamber 1050, stretching the diaphragm 1035 until contact is made with the distal end 1040 of the dispensing device 1000. Through this motion, the diaphragm 1035 and septum 1070 are pierced by puncturing end 1075 of the administration element 1080. The resultant pressure differential that exists between the storage volume 1066 and the ambient environment allows the plunger rod 1055 and stopper 1060 to translate distally into the body of the primary container 1025, expelling the beneficial agent 1065 through the fluid passage of the administration element 1080, entering through the puncturing end 1075 and ejecting through the distal port 1085.

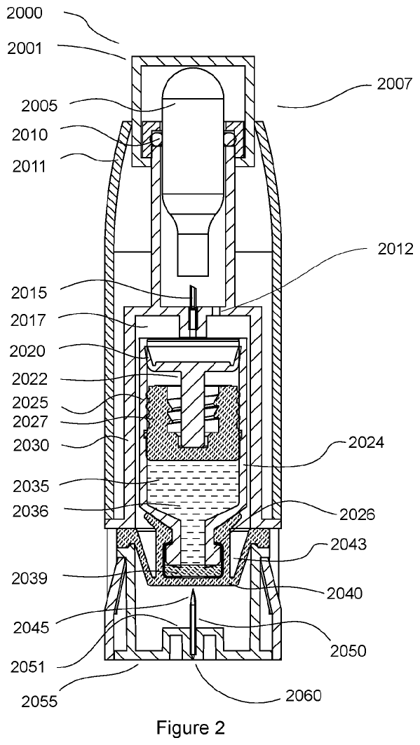

FIG. 2 illustrates a cross-sectional view of a dispensing device 2000 comprising a jet injector in an inactive state prior to activation. The dispensing device 2000 includes a proximal end 2007 and a distal end 2055 and may include an activator 2001 at the proximal end 2007. Actuation of the activator 2001 may be facilitated by a proximal retaining element 2010 which may include ball bearings, wheels, gears, a gasket, or a pin and may optionally provide resistance to movement of the activator 2001. A pressurized fluid source 2005 may be positioned in such a way that downward force, applied to the activator 2001, that the pressurized fluid source 2005 will be forced to come into contact with a fluid source initiator 2015, puncturing the pressurized fluid source 2005. Upon breach of the pressurized fluid source 2005, the pressurized gas will fill the space and travel through a passage 2012 into a pressure chamber 2017 distal to the pressurized fluid source 2005. The pressure chamber includes walls that form an inner barrel 2030, situated within the body 2011, that may encompass a primary container 2025. The distal end of the inner barrel may terminate against the diaphragm 2040, which may provide a fluid-tight seal. The diaphragm 2040 may comprise a flexible elastomeric material that seals the pressure chamber 2017. The diaphragm may connect and hold the distal end of the primary container 2025.

As shown in FIG. 2, the primary container 2025 may be situated within the pressure chamber 2017 and engaged with the diaphragm 2040. The diaphragm 2040 may include a receptacle 2043 with which the primary container 2025 is engaged, and the primary container 2025 may optionally include a fitment 2026 where it engages with the receptacle 2043. The primary container 2025 may include a storage volume 2035 for storing a beneficial agent 2036 and may be configured to deplete the beneficial agent 2036 through a distal port 2060 located at the distal end 2055 when a pressurized fluid is provided to the pressure chamber 2017. In one variation, the primary container 2025 may be a flexible cartridge configured to collapse in the active state when a pressurized fluid is provided into the pressure chamber 2017. In another variation, the primary container 2025 may be a rigid cartridge which can include a rigid sidewall 2024, a plunger 2020, optionally including a plunger rod 2022, and/or a stopper 2027 which may be configured to bound the storage volume 2035 against the volume of the pressure chamber 2017 and/or an interstitial volume between the plunger 2020 and stopper 2027. A rigid cartridge may include a distal end with a septum 2039 pierceable by a puncturing end 2045 of an administration element 2050.

Accumulating pressure within the pressure chamber 2017 is applied to a plunger 2020 positioned at the proximal end of the primary container 2025. The resultant pressure on the plunger 2020 induces distal translation of the primary container 2025 within the pressure chamber 2017 within the confines of the inner barrel 2030 until coming to rest on an inner wall of the distal end 2055 of the dispensing device 2000. As the pressurized fluid source 2005 is forced downward, the diaphragm 2040 of the pressure chamber 2017 and septum 2039 of the primary container 2025 are pierced by the puncturing end 2045 of an administration element 2050, secured by an administration element hub 2051, in such a manner as to create a pressure differential between the storage volume 2035 of the primary container 2025 which may contain a beneficial agent 2036, but that does not break the seal of the pressure chamber 2017. Continued pressure on the plunger 2020 may result in the distal translation of a plunger rod 2022 and stopper 2027 within the primary container 2025, causing the expulsion of the beneficial agent 2036 through the fluid passageway of the administration element 2050, entering through the puncturing end 2045 and exiting through the distal port 2060 at the distal end 2055 of the dispensing device 2000.

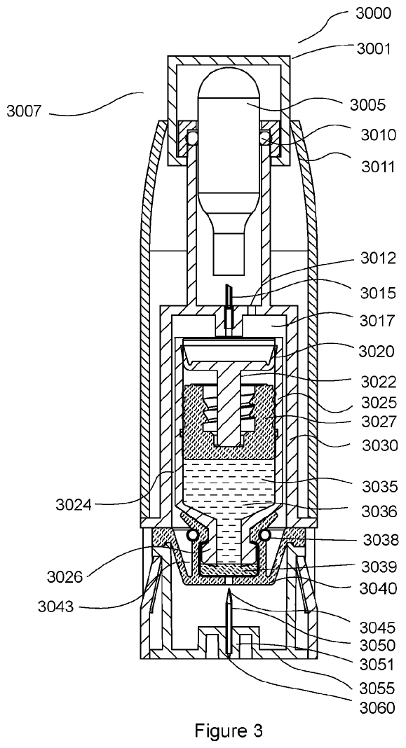

FIG. 3 illustrates a cross-sectional view of an inactive state of a dispensing device 3000 comprising a jet injector pre-activation. The dispensing device 3000 includes a rigid body 3011 with an activator 3001 at the proximal end 3007. The dispensing device 3000 includes a proximal end 3007 containing a pressurized fluid source 3005, which is located distally relative to the activator 3001, as well as a pressure chamber 3017 situated distal to the pressurized fluid source 3005 and separated by a rigid wall with an entry passage 3012. Within the pressure chamber 3017, the dispensing device 3000 may include a primary container 3025 that may hold beneficial agent 3036 within a storage volume 3035. The pressure chamber 3017 may comprise a sidewall 3030 and a diaphragm 3040 made of a flexible elastomeric material that is situated towards the distal end 3055 of the dispensing device 3000. This diaphragm 3040 encompasses the nozzle of the primary container 3025 and is secured by sets of seal assist springs 3038. The storage volume of the primary container 3025 may be defined by a rigid wall, a septum 3039 located at the distal end, and a plunger 3020 positioned between the proximal end of the primary container 3025 and the pressure chamber 3017. The plunger 3020 is connected to a plunger rod 3022, which may drive the downward translation of a stopper 3027 located within the primary container 3025. Each region is oriented along the central vertical axis of the rigid body 3011 of the dispensing device 3000.

As shown in FIG. 3, the primary container 3025 may be situated within the pressure chamber 3017 and engaged with the diaphragm 3040. The diaphragm 3040 may include a receptacle 3043 with which the primary container 3025 is engaged, and the primary container 3025 may optionally include a fitment 3026 where it engages with the receptacle 3043. The primary container 3025 may include a storage volume 3035 for storing a beneficial agent 3036 and may be configured to deplete the beneficial agent 3036 through a distal port 3060 located at the distal end 3055 when a pressurized fluid is provided to the pressure chamber 3017. In one variation, the primary container 3025 may be a flexible cartridge configured to collapse in the active state when a pressurized fluid is provided into the pressure chamber 3017. In another variation, the primary container 3025 may be a rigid cartridge which can include a rigid sidewall 3024, a plunger 3020, optionally including a plunger rod 3022, and/or a stopper 3027 which may be configured to bound the storage volume 3035 against the volume of the pressure chamber 3017 and/or an interstitial volume between the plunger 3020 and stopper 3027. A rigid cartridge may include a distal end with a septum 3039 pierceable by a piercing end of an administration element 3050.

Activation of the activator 3001 may cause translation of the pressurized fluid source 3005, facilitated by a retaining element 3010 which may include bearings, wheels, gears, a gasket, and/or a pin that may be of low or high resistance, towards a fluid source initiator 3015. Upon initiation of the pressurized fluid source 3005 by the fluid source initiator 3015, a pressurized fluid provided by the pressurized fluid source 3005 fills the proximal end 3007 and flows into the pressure chamber 3017 through an entry passage 3012. The resultant pressure of the pressurized fluid may apply a force upon the plunger 3020 that causes distal translation of the primary container 3025. The primary container 3025 continues downward movement until coming to rest upon the inner wall of the distal end 3055 of the dispensing device 3000. In doing so, the penetrating end 3045 of an administration element 3050, held in place by the supporting administration element hub 3051, passes through the diaphragm 3040 and punctures the septum 3039 of the primary container 3025. The pressure differential existing between the pressure chamber 3017 and the ambient environment enhances the displacement of the beneficial agent 3036 within the storage volume 3035 of the primary container 3025. This solution is dispensed at high velocity through the administration element 3050 and out the distal port 3060 due to the high-pressure present in the pressure chamber 3017 that applies a downward force to the plunger 3020, plunger rod 3022, and stopper 3027 into the primary container 3025.

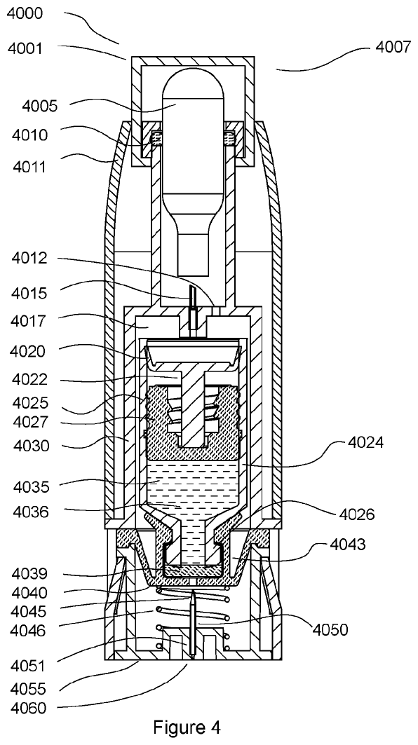

FIG. 4 illustrates a cross-sectional view of the inactive state of a dispensing device 4000 comprising a jet injector pre-activation. The dispensing device 4000 may include an activator 4001 proximal to the body 4011 defined by a rigid wall 4030. The body 4011 may be comprised of three regions: a pressurized fluid source 4005, a pressure chamber 4017, and a primary container 4025. The pressurized fluid source 4005, containing pressurized gas, may be encompassed within, distal to, or a combination of these in orientation to the activator 4001. A fluid source initiator 4015 may be situated distal to the pressurized fluid source 4005. The proximal end may include rigid walls and an entry passage 4012, providing fluid communication to the pressure chamber 4017 (adjacently positioned). The pressure chamber is located distally in relation to the chamber containing the pressurized fluid source 4005 and may be defined by a combination of rigid walls and a diaphragm 4040 (e.g., a flexible diaphragm) comprised of an elastomeric material towards the distal end and distal end 4055 of the dispensing device 4000. A primary container 4025, containing a storage volume 4035 that may hold a beneficial agent 4036 may be disposed within the central barrel of the pressure chamber, held in place by the surrounding walls and connected to the diaphragm 4040. The proximal end of the primary container 4025 is sealed by a stopper 4027 that is driven by a plunger rod 4022 and a plunger 4020 associated therewith that is exposed to the pressure chamber 4017. The distal end of the primary container 4025 is sealed by a septum 4039 that may be comprised of an elastomeric material. The primary container 4025 may be maintained in the initial position in response to the forces of the diaphragm 4040 and a biasing element 4046, such as a retraction spring positioned distally to the assembly, such that an increase in pressure within the pressure chamber 4017 may be required to overcome the forces and allow translation of the primary container 4025 towards the distal end 4055 where an administration element 4050 (e.g., with a hollow portion), that may serve as a liquid passage, is housed at least partly within an administration element hub 4051.

As shown in FIG. 4, the primary container 4025 may be situated within the pressure chamber 4017 and engaged with the diaphragm 4040. The diaphragm 4040 may include a receptacle 4043 with which the primary container 4025 is engaged, and the primary container 4025 may optionally include a fitment 4026 where it engages with the receptacle 4043. The primary container 4025 may include a storage volume 4035 for storing a beneficial agent 4036 and may be configured to deplete the beneficial agent 4036 through a distal port 4060 located at the distal end 4055 when a pressurized fluid is provided to the pressure chamber 4017. In one variation, the primary container 4025 may be a flexible cartridge configured to collapse in the active state when a pressurized fluid is provided into the pressure chamber 4017. In another variation, the primary container 4025 may be a rigid cartridge which can include a rigid sidewall 4024, a plunger 4020, optionally including a plunger rod 4022, and/or a stopper 4027 which may be configured to bound the storage volume 4035 against the volume of the pressure chamber 4017 and/or an interstitial volume between the plunger 4020 and stopper 4027. A rigid cartridge may include a distal end with a septum 4039 pierceable by a piercing end 4045 of an administration element 4050.

Activation of the activator 4001 may drive distal movement of the pressurized fluid source 4005, which may be facilitated by a proximal retaining element 4010 that may include the ball bearings, gears, wheels, or a gasket and may provide low or high resistance, towards the fluid source initiator 4015. Initiation of the pressurized fluid source 4005 causes pressurized fluid to be provided to the proximal end 4007 and passes into the pressure chamber 4017 through the entry passage 4012 and causes the displacement of the primary container 4025 from its pre-activation position due to an increase in uniformly distributed force upon the plunger 4020. This causes distal translation of the primary container 4025, stretching the diaphragm 4040 and collapsing the biasing element 4046 until the primary container 4025 reaches the furthest possible position, resting against the distal end 4055 of the dispensing device 4000. In this process, an administration element 4050, including a piercing end 4045, may pass through an opening in the diaphragm 4040 and pierce the septum 4039 that seals the primary container 4025, creating a fluid path between the storage volume 4035 and the external environment through the distal port 4060 at the distal end 4055 of the dispensing device 4000. The relief of pressure within the primary container 4025 facilitates the translation of the stopper 4027, driven by the internal forces of the pressure chamber 4017 acting upon the plunger 4020, into the primary container 4025. Increasing pressure and decreasing volume cause the expulsion of the beneficial agent 4036 through the administration element 4050.

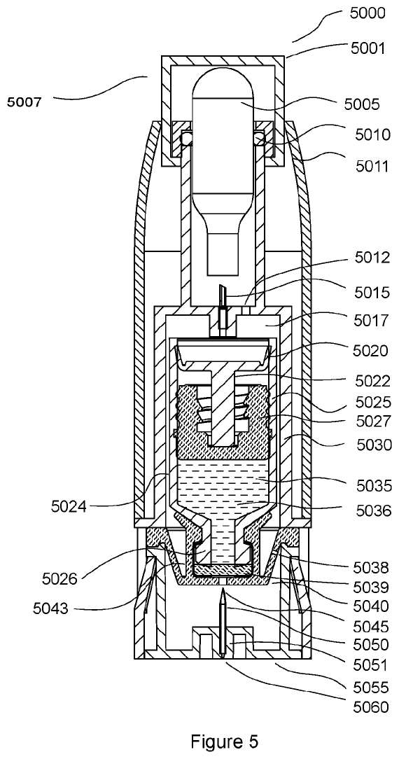

FIG. 5 illustrates a cross-sectional view of the initial pre-activation state of a dispensing device 5000 comprising a jet injector in the inactive state. The dispensing device 5000 may comprise a rigid body 5011 with an activator 5001 at the proximal end 5007. A pressurized fluid source 5005 may be disposed relative to the activator 5001, such that the application of downward force causes distal translation of the pressurized fluid source 5005, which may be facilitated by a retaining element 5010, including bearings, wheels, gears, and/or a gasket, towards a fluid source initiator 5015. Upon initiation, the pressurized fluid source 5005 may provide a pressurized fluid that may flow through an entry passage 5012 into the pressure chamber 5017 situated distally relative to the pressurized fluid source 5005. The pressure chamber 5017 may be comprised of rigid walls 5030 and a diaphragm 5040 (e.g., flexible diaphragm) of elastomeric material. The diaphragm may comprise a vent hole 5038, serving as a passageway between the pressure chamber 5017 and the lower region of the rigid body 5011. A primary container 5025 containing a storage volume 5035 that may hold a beneficial agent 5036 may be disposed within the central barrel of the pressure chamber 5017. The proximal end of the primary container 5025 may be sealed by a plunger 5020 and stopper 5027, connected by a plunger rod 5022. The distal end of the primary container 5025, towards the distal end 5055 of the dispensing device 5000, is engaged with and may be partially or wholly surrounded by a receptacle 5043 of the diaphragm 5040 within the pressure chamber 5017.

As shown in FIG. 5, the primary container 5025 may be situated within the pressure chamber 5017 and engaged with the diaphragm 5040. The diaphragm 5040 may include a receptacle 5043 with which the primary container 5025 is engaged, and the primary container 5025 may optionally include a fitment 5026 where it engages with the receptacle 5043. The primary container 5025 may include a storage volume 5035 for storing a beneficial agent 5036 and may be configured to deplete the beneficial agent 5036 through a distal port 5060 located at the distal end 5055 when a pressurized fluid is provided to the pressure chamber 5017. In one variation, the primary container 5025 may be a flexible cartridge configured to collapse in the active state when a pressurized fluid is provided into the pressure chamber 5017. In another variation, the primary container 5025 may be a rigid cartridge which can include a rigid sidewall 5024, a plunger 5020, optionally including a plunger rod 5022, and/or a stopper 5027 which may be configured to bound the storage volume 5035 against the volume of the pressure chamber 5017 and/or an interstitial volume between the plunger 5020 and stopper 5027. A rigid cartridge may include a distal end with a septum 5039 pierceable by a piercing end of an administration element 5050.

Accumulating pressure within the pressure chamber 5017 displaces the primary container 5025 in a downward direction. This pressure may be controllably relieved through the vent hole 5038 to equalize pressure between the pressure chamber 5017 and the lower region of the rigid body 5011 to reduce the overall forces acting upon the plunger 5020 and, therefore, upon the primary container 5025. Distal translation of the primary container results in penetration of the septum 5039 by a puncturing end 5045 of an administration element 5050 (e.g., with a hollow portion), held in place by an administration element hub 5051, creating a liquid passageway. The pressure differential that exists between the pressure chamber 5017, the storage volume 5035 of the primary container 5025, and the ambient environment causes the plunger 5020 and plunger rod 5022 to drive the stopper 5027 into the storage volume of the primary container 5025, leading to the expulsion of the beneficial agent 5036 at high velocity through the administration element 5050 and the distal port 5060 at the distal end 5055 of the dispensing device 5000.

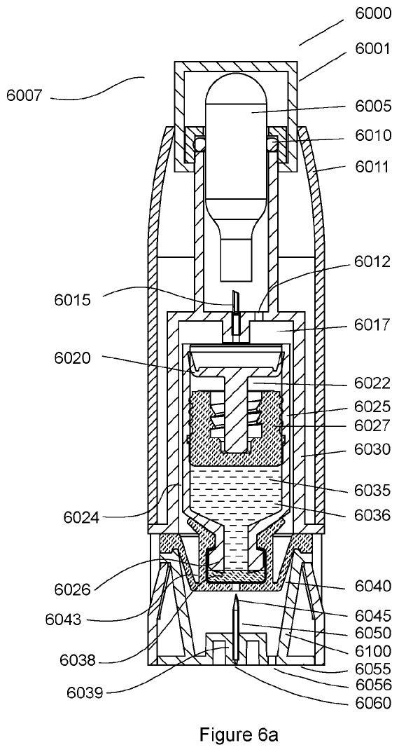

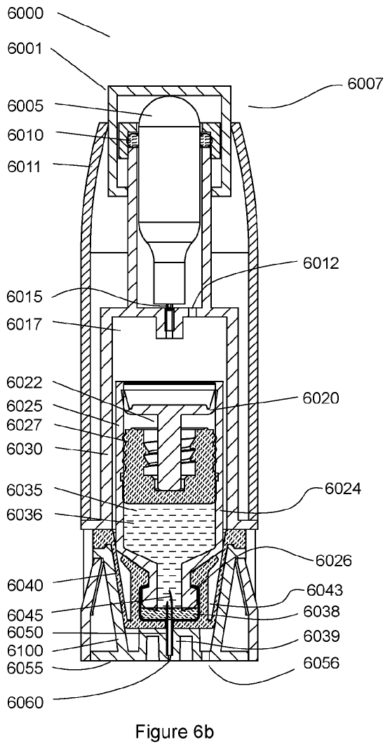

FIGS. 6a-6b illustrates a cross-sectional view of a dispensing device 6000 comprising a jet injector. The dispensing device 6000 includes an activator 6001 (e.g., a proximal activator) and a rigid body 6011. FIG. 6a depicts the injector in its pre-activation state, showing many of the internal features. A pressurized fluid source 6005 may be positioned distal to the activator 6001, in such a way that downward force upon the activator 6001 displaces the pressurized fluid source 6005 through a retaining element 6010 that may be of low or high resistance and causes the pressurized fluid source 6005 to interact with a fluid source initiator 6015, resulting in a pressurized fluid being provided to the proximal end 6007 of the dispensing device 6000. An entry passage 6012 may permit the transmission of pressurized fluid into a pressure chamber 6017. In the initial state, a primary container 6025 disposed within the sidewall 6030 of the pressure chamber 6017 may be sealed against the volume of the pressure chamber by an assembly that may include a plunger 6020, a plunger rod 6022, and a stopper 6027 at the proximal end of the primary container 6025. The primary container 6025 may comprise a storage volume 6035 disposed distally relative to the stopper. The storage volume 6035 may contain a beneficial agent 6036. The distal end of the primary container 6025 may comprise a septum 6038, which may be formed of an elastomeric material that creates a seal upon the primary container 6025. The primary container 6025 may be partially or entirely enveloped by a diaphragm 6040, which may be made of flexible elastomeric material. The diaphragm 6040 may seal the pressure chamber 6017 in the inactive and/or active state. An administration element hub 6039 may be positioned along the interior surface of the distal end 6055 of the dispensing device 6000 in such a manner that the administration element 6050 may protrude towards the septum 6038. A pressure relief vent 6056 may be provided at the distal end 6055. The pressure relief vent 6056 may controllably release accumulated pressure in response to the distal movement of the diaphragm 6040, thus allowing the primary container 6025 to be displaced distally.

As shown in FIGS. 6a & 6b, the primary container 6025 may be situated within the pressure chamber 6017 and engaged with the diaphragm 6040. The diaphragm 6040 may include a receptacle 6043 with which the primary container 6025 is engaged, and the primary container 6025 may optionally include a fitment 6026 where it engages with the receptacle 6043. The primary container 6025 may include a storage volume 6035 for storing a beneficial agent 6036 and may be configured to deplete the beneficial agent 6036 through a distal port 6060 located at the distal end 6055 when a pressurized fluid is provided to the pressure chamber 6017. In one variation, the primary container 6025 may be a flexible cartridge configured to collapse in the active state when a pressurized fluid is provided into the pressure chamber 6017. In another variation, the primary container 6025 may be a rigid cartridge which can include a rigid sidewall 6024, a plunger 6020, optionally including a plunger rod 6022, and/or a stopper 6027 which may be configured to bound the storage volume 6035 against the volume of the pressure chamber 6017 and/or an interstitial volume between the plunger 6020 and stopper 6027. A rigid cartridge may include a distal end with a septum 6038 pierceable by a piercing end of an administration element 6050.

FIG. 6b illustrates a design of a dispensing device 6000 comprising a jet injector, shown in the active state where pressurization of the pressure chamber 6017 occurs by means of puncturing the pressurized fluid source 6005. Increasing, uniform pressure is applied to the plunger 6020, sealing the proximal end of the primary container 6025 as well as the diaphragm 6040 that seals the distal end of the pressure chamber 6017. The diaphragm 6040 may be configured in such a way that upon an increase in pressure, the diaphragm 6040 may extend distally, relative to its resting state, along support walls 6100 disposed within the lower region of the rigid body 6011, translating the primary container 6025 with the diaphragm 6040 until coming to rest on the interior surface of the distal end 6055. As a consequence of its downward movement, the septum 6038 of the primary container 6025 may be pierced by the puncturing end 6045 of the administration element 6050, resulting in a pressure differential that forms between the pressure chamber 6017, storage volume 6035 of the primary container 6025, and ambient environment. The plunger 6020 drives the distal translation of the stopper 6027, expelling the beneficial agent 6036 through the fluid passageway of the administration element 6050 and the distal port 6060 at high velocity.

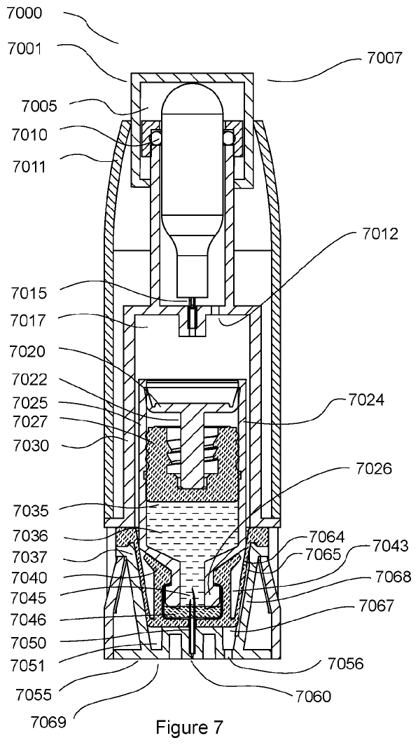

FIG. 7 illustrates a cross-sectional view of a dispensing device 7000 comprising a jet injector in the active state. The dispensing device 7000 may include a rigid body 7011 with an activator 7001 at the proximal end 7007. FIG. 7 illustrates a configuration in which the dispensing device 7000 includes a region containing a pressurized fluid source 7005 retained by a retaining element 7010 and situated distal to the activator 7001. The pressurized fluid source 7005 may provide a pressurized fluid into the proximal end 7007 when initiated by a fluid source initiator 7015, illustrated in FIG. 7 as a pressurized gas canister punctured by a pin. The dispensing device 7000 includes a pressure chamber 7017 (e.g., centrally located within the dispensing device 7000) and a primary container 7025 within the sidewall 7030 of the pressure chamber 7017. The primary container 7025 may include any of a plunger 7020, plunger rod 7022, or stopper 7027 at the proximal end and/or a septum 7046 that may be made of an elastomeric material that seals the distal end. Elements of the primary container 7025 may enclose a storage volume 7035 that may contain a beneficial agent 7036. The distal end of the primary container 7025 may be attached, either in whole or in part, to a diaphragm 7040 (e.g., a flexible, elastomeric diaphragm) enclosing the distal end of the pressure chamber 7017.

As shown in FIG. 7, the primary container 7025 may be situated within the pressure chamber 7017 and engaged with the diaphragm 7040. The diaphragm 7040 may include a receptacle 7043 with which the primary container 7025 is engaged, and the primary container 7025 may optionally include a fitment 7026 where it engages with the receptacle 7043. The primary container 7025 may include a storage volume 7035 for storing a beneficial agent 7036 and may be configured to deplete the beneficial agent 7036 through a distal port 7060 located at the distal end 7055 when a pressurized fluid is provided to the pressure chamber 7017. In one variation, the primary container 7025 may be a flexible cartridge configured to collapse in the active state when a pressurized fluid is provided into the pressure chamber 7017. In another variation, the primary container 7025 may be a rigid cartridge which can include a rigid sidewall 7024, a plunger 7020, optionally including a plunger rod 7022, and/or a stopper 7027 which may be configured to bound the storage volume 7035 against the volume of the pressure chamber 7017 and/or an interstitial volume between the plunger 7020 and stopper 7027. A rigid cartridge may include a distal end with a septum 7046 pierceable by a piercing end of an administration element 7050.

A downward force applied to the activator 7001 induces distal translation of the pressurized fluid source 7005 towards a puncturing pin positioned distally in relation to the pressurized fluid source 7005. Escaping gas may pass through a passageway 7012 into the pressure chamber 7017 disposed distally in relation to the pressurized fluid source 7005. The increase in pressure drives the primary container 7025 and assembly comprising the plunger 7020, plunger rod 7022, and stopper 7027 towards the distal end 7055 of the dispensing device 7000, stretching the diaphragm 7040 in the process. The elongation of the diaphragm 7040 follows the structural walls 7037 of the lower region of the rigid body 7011. Upon reaching the interior wall of the distal end 7055, the septum 7046 of the primary container 7025 is punctured by the piercing end 7045 of an administration element 7050, held in place at the administration element hub 7051, creating a pressure differential between the primary container 7025 and pressure chamber 7017 and that of the surrounding environment. Continued pressure upon the plunger 7020 drives the stopper 7027 into the storage volume of the primary container 7025, ejecting the beneficial agent 7036 through distal port 7060 at the distal end 7055 of the dispensing device 7000. Excess pressure within the pressure chamber 7017 may escape through a diaphragm vent 7064, which may be located in the diaphragm 7040 that aligns with a fluid passage 7065 comprising an adjustable bleed channel, while the pressure within the lower chamber of the dispensing device 7000 that accumulates due to the expansion of the diaphragm 7040 is forced through a pressure relief vent 7056. In turn, the configuration and sizing of the diaphragm vent 7064, fluid passage 7065, and pressure relief vent 7056 may be used to control the pressure differentials in the various compartments of the dispensing device 7000 to assist in the control of the rates at which the primary container 7025 moves and at which the beneficial agent 7036 is dispensed from the dispensing device 7000.

The dispensing device 7000 may include a fluid passage 7065. The arrangement may be such that a fluid flow of the pressurized fluid may flow through the fluid passage 7065 from the pressure chamber when a pressurized fluid is provided to the pressure chamber 7017. The fluid passage 7065 may provide for fluid flow through the diaphragm 7040. This fluid flow may flow through a diaphragm vent 7064, which may comprise a bleed hole. The primary container 7025 may be configured to retract proximally when the pressurized fluid is expelled from the pressure chamber 7017 through the fluid passage 7065. A dwell time during which the dispensing device 7000 may remain in the active state may be defined by a retraction force acting on the primary container 7025 and/or by a flow rate of the fluid flow through the fluid passage 7065. The storage volume 7035 of the primary container 7025 may be in fluid communication with an external environment external to the dispensing device 7000 during substantially the entire dwell time. A distal chamber 7067 may be provided and may be disposed on the distal side of the diaphragm 7040 and within the dispensing device 7000. The volume of the distal chamber 7067 may be defined by one or more distal chamber walls, which may include a distal chamber sidewall 7068 and/or a distal wall 7069. A proximal bound of the distal chamber 7067 may be provided by the diaphragm 7040. The distal chamber 7067 may be adapted such that the fluid flow from the pressure chamber 7017 through the fluid passage 7065 may be directed into the distal chamber 7067 in the active state.

The arrangement of the dispensing device 7000 may be such that the primary container 7025 can retract proximally when the pressure of the pressurized fluid within the pressure chamber 7017 is lower than a threshold pressure. The dispensing device 7000 may include a biasing element such as a spring, and the biasing element may provide a force on the primary container 7025, biasing it in the proximal direction. The diaphragm 7040 may optionally be adapted to bias the primary container 7025 to retract proximally, such as by forming a diaphragm 7040 from an elastomeric material with a defined elastic modulus. The primary container 7025 may be configured to retract proximally when the pressure of the pressurized fluid in the pressure chamber 7017 is lower than a threshold pressure. The threshold pressure and/or total biasing force may be provided, defined, and/or modulated by the pressure of the pressurized fluid within the distal chamber 7067, a biasing element that may be disposed at the distal end of the dispensing device 7000, and/or the diaphragm 7040 in any combination.

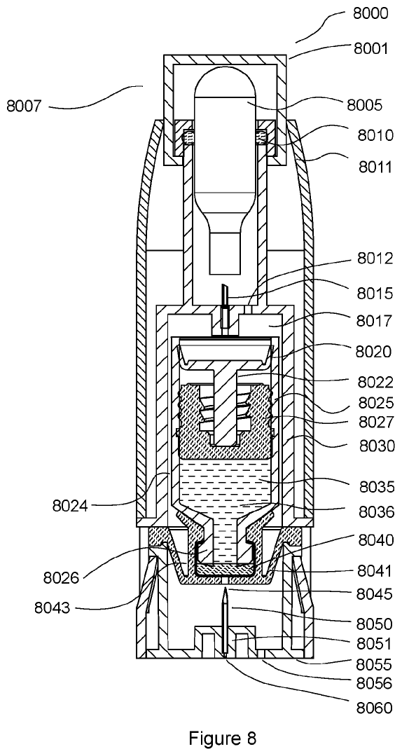

FIG. 8 illustrates a cross-sectional view of a dispensing device 8000 comprising a jet injector in the pre-activation inactive state. The dispensing device 8000 may comprise an activator 8001 proximal to the body 8011, wherein each may be defined by a rigid wall. The body 8011 may be comprised of three regions: a pressurized fluid source 8005, a pressure chamber 8017, and a primary container 8025. The pressurized fluid source 8005 may be positioned distal to, partially, or wholly encompassed by the activator 8001 and may be retained by a proximal retaining element 8010 which may include ball bearings, gears, wheels, gaskets, and/or pins and which may provide resistance to distal movement of the pressurized fluid source 8005. Through activation of the activator 8001, as by application of downward force to a button, the pressurized fluid source 8005 may be moved distally to interact with a fluid source initiator 8015. Initiation of the pressurized fluid source 8005 may cause a pressurized fluid to be provided to a proximal end 8007 of the dispensing device 8000. The pressurized fluid may flow through an entry passage 8012 into the pressure chamber 8017.

Disposed within the body 8011, the pressure chamber 8017 includes a sidewall 8030 optionally sealed at the distal end by a diaphragm 8041 that may be made of an elastomeric material. As shown in FIG. 8, the primary container 8025 may be situated within the pressure chamber 8017 and engaged with the diaphragm 8041. The diaphragm 8041 may include a receptacle 8043 with which the primary container 8025 is engaged, and the primary container 8025 may optionally include a fitment 8026 where it engages with the receptacle 8043. The primary container 8025 may include a storage volume 8035 for storing a beneficial agent 8036 and may be configured to deplete the beneficial agent 8036 through a distal port 8060 located at the distal end 8055 when a pressurized fluid is provided to the pressure chamber 8017. The distal end 8055 may include a vent 8056, providing fluid communication between the volume of a distal chamber and an ambient environment. In one variation, the primary container 8025 may be a flexible cartridge configured to collapse in the active state when a pressurized fluid is provided into the pressure chamber 8017. In another variation, the primary container 8025 may be a rigid cartridge which can include a rigid sidewall 8024, a plunger 8020, optionally including a plunger rod 8022, and/or a stopper 8027 which may be configured to bound the storage volume 8035 against the volume of the pressure chamber 8017 and/or an interstitial volume between the plunger 8020 and stopper 8027. A rigid cartridge may include a distal end with a septum 8040 pierceable by a piercing end of an administration element 8051.

A primary container 8025 containing a beneficial agent 8036 within a storage volume 8035 and sealed proximally by a stopper 8027 and distally by a septum 8040 may be present within the sidewall 8030 of the pressure chamber 8017 and connected at its distal end to the diaphragm 8041 that seals the pressure chamber 8017. As pressure accumulates in the pressure chamber 8017, the primary container 8025 is translated distally along with the diaphragm 8041 until it encounters the inner surface of the distal end 8055 of the dispensing device 8000. The primary container 8025 may include a plunger 8020 and a septum 8040, which may be made of an elastomeric material. When the distal movement of the primary container 8025 terminates, the septum 8040 of the primary container 8025 may be pierced by the piercing end 8045 of an administration element 8050 containing a fluid passageway, resulting in fluid communication between the storage volume 8035 and the external environment. Providing a fluid into the pressure chamber 8017 may apply pressure to the plunger 8020, driving the downward translation of the stopper 8027 through the plunger rod 8022, and causing the storage volume 8035 to deplete its contents (e.g., the beneficial agent 8036) to expel the contents via the distal port.

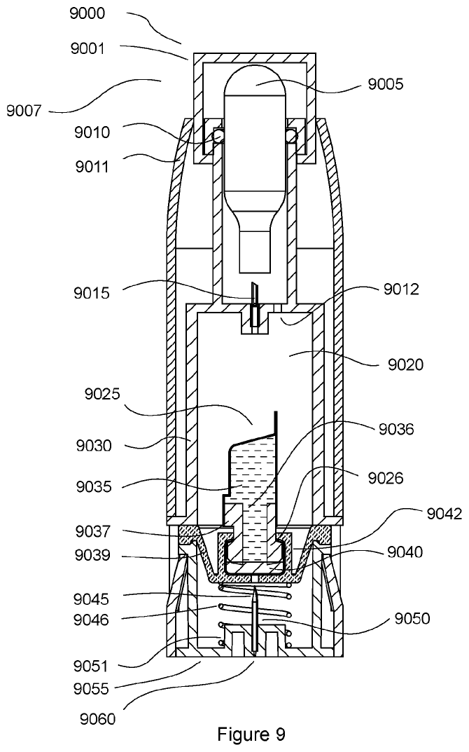

FIG. 9 illustrates a cross-sectional view of an inactive state of a dispensing device 9000, shown here as a jet injector with a primary container 9025, including a flexible cartridge. The dispensing device 9000 may include an activator 9001 proximal to the body 9011, and each may be defined by a rigid wall. A pressurized fluid source 9005, such as a gas canister, gas-generating chemical reaction, or pump, may be partially disposed within the body 9011. The body 9011 may include a pressure chamber 9020 and the primary container 9025. The pressurized fluid source 9005 may be disposed beneath and/or partially surrounded by a part of the activator 9001 and may be proximally positioned relative to a fluid source initiator 9015. An entry passage 9012 may be included. Such an entry passage 9012 can provide fluid communication between a proximal end 9007 of the dispensing device 9000 and the pressure chamber 9020. The pressure chamber 9020 may be disposed distal to the proximal end 9007 and may be defined by a sidewall 9030, which can take the form of a hollow cylinder. The pressure chamber 9020 may be sealed by a diaphragm 9039 (e.g., a flexible diaphragm) which can be located at a distal end of the pressure chamber 9020 and may be comprised of an elastomeric material. The dispensing device 9000 includes a distal end 9055.

The primary container 9025 may include a storage volume 9035, which can store a beneficial agent 9036. The primary container 9025 may be disposed within the pressure chamber 9020 and may be engaged to the diaphragm 9039 at the distal end. The diaphragm 9039 may include a receptacle 9042, which envelops a distal portion of the primary container 9025, that distal portion optionally including a fitment 9037 and a septum 9040. The septum 9040 may be composed of an elastomeric or thin rigid material. The primary container 9025 may be exposed to the forces of a pressurized fluid in the pressure chamber 9020, and the position of the primary container 9025 in the inactive state may be maintained by a biasing force of a biasing element 9046 (e.g., a spring), which biases the primary container 9025 in the proximal direction. An administration element 9050 may be provided and may include a fluid path and/or piercing end 9045. The biasing element 9046 may be disposed within an administration element hub 9051, optionally situated on an interior surface at the distal end 9055 of the dispensing device 9000. The administration element 9050 may be poised such that the piercing end 9045 punctures the septum 9040 when the dispensing device 9000 transitions from the inactive state to the active state, providing fluid communication between the storage volume 9035 and an external environment.

The primary container 9025 may include a flexible cartridge. The transition of the dispensing device 9000 between the active state and the inactive state may include flexing of the flexible cartridge, which flexing may be responsive to providing a pressurized fluid into the pressure chamber 9020 and/or penetration of the storage volume 9035 of the flexible cartridge by the administration element 9050. In the active state of the dispensing device 9000, the flexible cartridge may flex, and flexing of the flexible cartridge may deplete the storage volume 9035 and may then expel the beneficial agent 9036 through the administration element 9050 and/or the distal port 9060 located at the distal end 9055.

As shown in FIG. 9, the primary container 9025 may be situated within the pressure chamber 9020 and engaged with the diaphragm 9039. The diaphragm 9039 may include a receptacle 9042 with which the primary container 9025 is engaged, and the primary container 9025 may optionally include a fitment 9026 where it engages with the receptacle 9042. The primary container 9025 may include a storage volume 9035 for storing a beneficial agent 9036 and may be configured to deplete the beneficial agent 9036 through a distal port 9060 located at the distal end 9055 when a pressurized fluid is provided to the pressure chamber 9020. In one variation, the primary container 9025 may be a flexible cartridge, which may optionally include a flat side and/or a formed side defining a storage volume 9035, configured to collapse in the active state when a pressurized fluid is provided into the pressure chamber 9020. In another variation, the primary container 9025 may be a rigid cartridge which can include a rigid sidewall, a plunger optionally including a plunger rod, and/or a stopper which may be configured to bound the storage volume against the volume of the pressure chamber 9020 and/or an interstitial volume between the plunger and stopper. A primary container may include a distal end with a septum 9040 pierceable by a piercing end of an administration element 9050.

The activator 9001 may be responsive to an application of force, and such response may cause the distal translation of the pressurized fluid source 9005 through a proximal retaining element 9010 (e.g., a gasket, ball bearings, or gearing), which may provide resistance to distal translation. The pressurized fluid source 9005 may move distally to interact with a fluid source initiator 9015, such as rupturing pin, voltage source, actuator, or the like. The pressurized fluid source 9005 may be punctured, activated, or opened such that it will provide a pressurized fluid. The pressurized fluid may be provided to a proximal end 9007 of the dispensing device 9000 and may flow through an entry passage 9012 to the pressure chamber 9020. The pressurized fluid in the pressure chamber 9020 may cause the dispensing device 9000 to transition to the active state. This transition may involve distal stretching of the diaphragm 9039 and/or compression of the biasing element 9046 until the primary container 9025 reaches the distal end 9055 of the dispensing device 9000. In this process, the septum 9040 may be penetrated by the piercing end 9045 of the administration element 9050. The pressure differential between the ambient environment and storage volume 9035 of the primary container 9025 may expel the beneficial agent 9036 through the fluid passageway of the administration element 9050 and through the distal end 9055 at the distal port 9060.

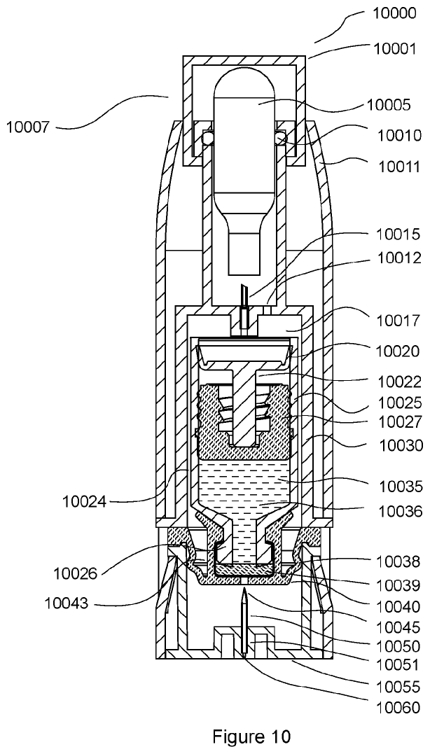

FIG. 10 depicts a cross-sectional view of a dispensing device 10000 comprising a jet injector in an inactive state pre-activation. The dispensing device 10000 may include an activator 10001 proximal to and/or disposed partly within a body 10011. Activation of the activator 10001 may cause distal translation of a pressurized fluid source 10005, facilitated by a proximal retaining element 10010 that may provide a resistance to motion, causing it to interact with a fluid flow initiator 10015. Initiation of the pressurized fluid source 10005 may provide a pressurized fluid that fills the space of the proximal end 10007 and flows through a passageway 10012 into a pressure chamber 10017 bounded by a sidewall 10030 and a diaphragm 10040. A primary container 10025 may be disposed within the pressure chamber 10017 and proximally sealed by a plunger 10020 with a plunger rod 10022 extending into an interior cavity of a stopper 10027. The distal end of the primary container 10025 may be sealed by a septum 10039 (e.g., an optionally elastomeric septum), and both may be engaged to the diaphragm 10040 of the pressure chamber 10017. The diaphragm 10040 may comprise stress relief joints 10038.

As shown in FIGS. 10, the primary container 10025 may be situated within the pressure chamber 10017 and engaged with the diaphragm 10040. The diaphragm 10040 may include a receptacle 10043 with which the primary container 10025 is engaged, and the primary container 10025 may optionally include a fitment 10026 where it engages with the receptacle 10043. The primary container 10025 may include a storage volume 10035 for storing a beneficial agent 10036 and may be configured to deplete the beneficial agent 10036 through a distal port 10060 located at the distal end 10055 when a pressurized fluid is provided to the pressure chamber 10017. In one variation, the primary container 10025 may be a flexible cartridge configured to collapse in the active state when a pressurized fluid is provided into the pressure chamber 10017. In another variation, the primary container 10025 may be a rigid cartridge which can include a rigid sidewall 10024, a plunger 10020, optionally including a plunger rod 10022, and/or a stopper 10027 which may be configured to bound the storage volume 10035 against the volume of the pressure chamber 10017 and/or an interstitial volume between the plunger 10020 and stopper 10027. A rigid cartridge may include a distal end with a septum 10039 pierceable by a piercing end of an administration element 10050.

Increasing pressure, due to the rupture of the pressurized fluid source 10005, is applied uniformly across the plunger 10020 and diaphragm 10040, resulting in the extension of the diaphragm 10040 and, with it, the primary container 10025, until coming to rest along the inner surface of the distal end 10055 of the dispensing device 10000. An administration element 10050 comprising a piercing end 10045 is held in place by an administration element hub 10051 along the central horizontal axes, projecting proximally in relation to the distal end 10055. Movement of the primary container 10025 to the distal end of the body 10011 results in the puncturing of the administration element 10050 into the storage volume 10035 of the primary container 10025 containing a beneficial agent 10036, creating a fluid passageway. Continued pressure along the proximal surface of the plunger 10020 compresses the storage volume 10035 by driving the stopper 10027 in the distal direction, expelling the beneficial agent 10036 at high velocity through the distal port 10060 of the administration element 10050.

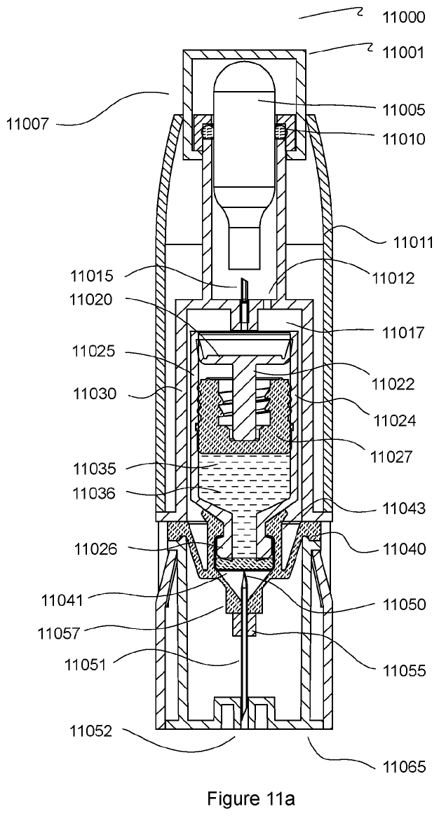

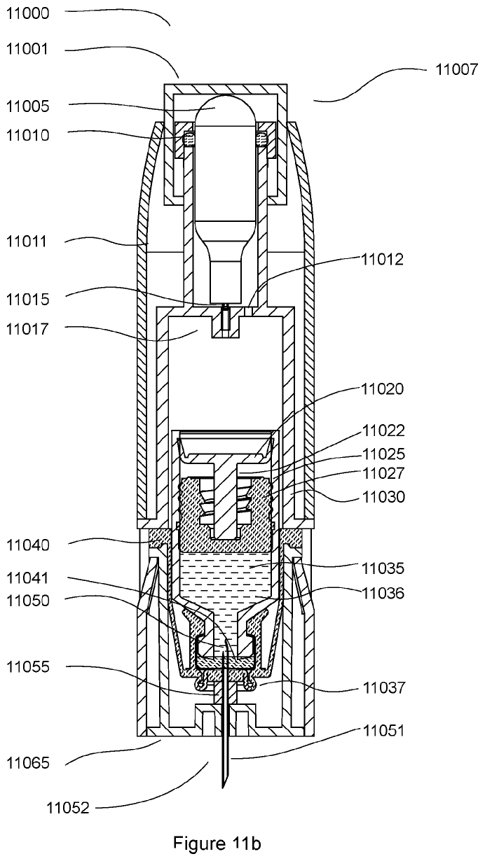

FIGS. 11a-b illustrates a cross-sectional view showing many of the internal components of a version of dispensing device 11000 comprising an auto-injector. The dispensing device 11000 may centrally include a body 11011 and may optionally include an activator 11001 positioned at the proximal end 11007. FIG. 11a shows the dispensing device 11000 in the inactive configuration, illustrating the pressurized fluid source 11005, which may be disposed partially within the activator 11001 and the body 11011 and retained in this position by a proximal retaining element 11010, such as a gasket, ball bearings, wheels, or gears. This proximal retaining element 11010 may also facilitate intentional displacement of the pressurized fluid source 11005. A fluid source initiator 11015, such as a rupturing pin, may be disposed in relation to the pressurized fluid source 11005 such that upon distal movement of the activator 11001, the pressurized fluid source 11005 will interact with the fluid source initiator 11015 such that the pressurized fluid source 11005 expels a pressurized fluid. This proximal assembly may optionally incorporate an entry passage 11012 that provides fluid communication of the pressurized into the pressure chamber 11017.

The pressure chamber 11017 may include a primary wall 11030 and a diaphragm 11040 made of a flexible material. A primary container 11025 comprising a rigid cartridge may be disposed within the pressure chamber 11017 and may include a beneficial agent 11036 stored within a storage volume 11035. The storage volume 11035 of the primary container 11025 may be defined by a stopper 11027 at a proximal end and a septum 11041 at a distal end, each of which may be composed of an elastomeric material. The primary container 11025 may include a plunger 11020 with a plunger rod 11022 disposed proximally relative to the stopper 11027. The distal end of the primary container 11025 is engaged to the diaphragm 11040 within the pressure chamber 11017 such that translation of the diaphragm 11040 will result in simultaneous distal displacement of the primary container 11025. An administration element 11051, including a piercing end 11050, may be disposed distal to the septum 11041 of the primary container 11025 and may be secured by an administration element hub 11055, which may be loosely affixed in the distal end 11065 of the body 11011. The administration element 11051 may be disposed partially within a distal port 11052 at the distal end 11065 of the dispensing device 11000. The diaphragm 11040 may include an administration element receptacle 11057, which may movably accommodate the administration element 11051, and the administration element receptacle 11057 may be located distal to the primary container 11025 at the distal end 11065 of the dispensing device 11000. A portion of the diaphragm 11040 may be adapted to deform responsive to the distal moving of the primary container 11025. The deformation may be such that the administration element 11051 and/or a piercing end 11050 thereof penetrates the primary container 11025.

As shown in FIGS. 1a-1b, the primary container 11025 may be situated within the pressure chamber 11017 and engaged with the diaphragm 11040. The diaphragm 11040 may include a receptacle 11043 with which the primary container 11025 is engaged, and the primary container 11025 may optionally include a fitment 11026 where it engages with the receptacle 11043. The primary container 11025 may include a storage volume 11035 for storing a beneficial agent 11036 and may be configured to deplete the beneficial agent 11036 through a distal port 11052 located at the distal end 11065 when a pressurized fluid is provided to the pressure chamber 11017. In one variation, the primary container 11025 may be a flexible cartridge configured to collapse in the active state when a pressurized fluid is provided into the pressure chamber 11017. In another variation, the primary container 11025 may be a rigid cartridge which can include a rigid sidewall 11024, a plunger 11020, optionally including a plunger rod 11022, and/or a stopper 11027 which may be configured to bound the storage volume 11035 against the volume of the pressure chamber 11017 and/or an interstitial volume between the plunger 11020 and stopper 11027. A rigid cartridge may include a distal end with a septum 11041 pierceable by a piercing end of an administration element 11051.

FIG. 11b illustrates the dispensing device 11000 in the active state. A downward force applied to the activator 11001 may cause distal movement of the pressurized fluid source 11005, causing the fluid source initiator 11015 to interact with the pressurized fluid source 11005 and provide a pressurized fluid which may flow through the entry passage 11012 into the pressure chamber 11017. The receptacle 11043 may be compressed against the primary container 11025 when a pressurized fluid is provided to the pressure chamber 11017, and the receptacle 11043 may be arranged to compress against a fitment 11026 included in the primary container 11025. Such a fitment 11026 may be disposed substantially within the primary container 11025 and may be a formed rigid shape. A retaining element may optionally be disposed relative to the receptacle and may be adapted to retain the receptacle 11043 against the primary container 11025. The retaining element may be adapted to maintain a seal between the receptacle 11043 and the primary container 11025.

The pressurized fluid may act on the plunger 11020 and/or diaphragm 11040 to cause distal movement of the primary container 11025. The translation of the primary container 11025 may cause the administration element hub 11055 to move in the distal direction. Upon dislocation of the administration element hub 11055, the moveable assembly, including the primary container 11025, administration element hub 11055, and administration element 11051, may move to the distal end 11065 of the body 11011. A section of the diaphragm 11040 may distort into a distorted section 11037. In this active state, the administration element 11051 may be deployed through the distal port 11052 at the distal end 11065 of the body 11011, and the piercing end 11050 may penetrate the septum 11041 and enter the storage volume 11035 of the primary container 11025. This arrangement may provide fluid communication such that the plunger 11020, plunger rod 11022, and stopper 11027 move distally within the primary container 11025 and expel the beneficial agent 11036 from the storage volume 11035 through the administration element 11051 the distal port 11052 into an external environment.

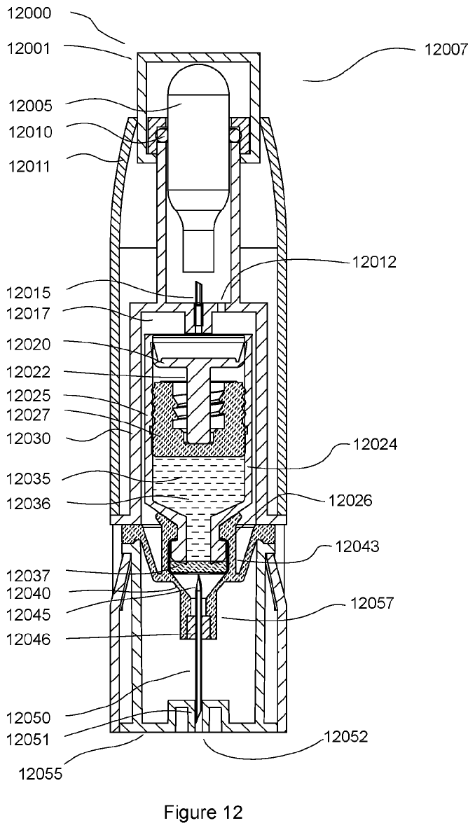

FIG. 12 embodies a cross-sectional view of a dispensing device 12000 comprising an auto-injector in a pre-activation inactive state. The dispensing device 12000 may include a body 12011 centrally disposed and a proximal end 12007 including an activator 12001. FIG. 12 illustrates a pressurized fluid source 12005, which may optionally be a pressurized gas canister, may be positioned relative to the activator 12001 such that activation may cause distal translation of the pressurized fluid source 12005. The movement of the pressurized fluid source 12005 may be facilitated by a proximal retaining element 12010 that may provide a resistance to movement. A fluid source initiator 12015 is disposed distal to the pressurized fluid source 12005, adjacent to an entry passage 12012 that permits gas exchange into a pressure chamber 12017 at the center of the body 12011. The pressure chamber 12017 may include sidewalls 12030 with rigid walls and a distal end terminating in a diaphragm 12040 that may be flexible and/or comprised of an elastomeric material. A primary container 12025 with a storage volume 12035 that may contain a beneficial agent 12036 is disposed within the sidewall 12030 and is sealed at the distal end by a septum 12037 and at the proximal end with a stopper 12027. The stopper 12027 is impregnated by the plunger rod 12022 and thus connected to a plunger 12020 that covers the proximal end of the primary container 12025. The distal neck of the primary container 12025 is held in place by the diaphragm 12040 of the pressure chamber 12017 in such a manner as to force downward translation of the primary container 12025 as the diaphragm 12040 extends.

As shown in FIG. 12, the primary container 12025 may be situated within the pressure chamber 12017 and engaged with the diaphragm 12040. The diaphragm 12040 may include a receptacle 12043 with which the primary container 12025 is engaged, and the primary container 12025 may optionally include a fitment 12026 where it engages with the receptacle 12043. The primary container 12025 may include a storage volume 12035 for storing a beneficial agent 12036 and may be configured to deplete the beneficial agent 12036 through a distal port 12052 located at the distal end 12055 when a pressurized fluid is provided to the pressure chamber 12017. In one variation, the primary container 12025 may be a flexible cartridge configured to collapse in the active state when a pressurized fluid is provided into the pressure chamber 12017. In another variation, the primary container 12025 may be a rigid cartridge which can include a rigid sidewall 12024, a plunger 12020, optionally including a plunger rod 12022, and/or a stopper 12027 which may be configured to bound the storage volume 12035 against the volume of the pressure chamber 12017 and/or an interstitial volume between the plunger 12020 and stopper 12027. A rigid cartridge may include a distal end with a septum 12037 pierceable by a piercing end of an administration element 12050.

The diaphragm 12040 may include an administration element receptacle 12057, which may movably accommodate the administration element 12050, and the administration element receptacle 12057 may be located distal to the primary container 12025 at the distal end 12055 of the dispensing device 12000. A portion of the diaphragm 12040 may be adapted to deform responsive to the distal moving of the primary container 12025. The deformation may be such that the administration element 12050 and/or a piercing end 12045 thereof penetrates the primary container 12025. A portion of the diaphragm 12040 encompasses the upper portion of an administration element 12050 that consists of a piercing end 12045 and a distal port 12052 and seals to an administration element hub 12046 (e.g., a loosely affixed administration element hub) flanking the body of the administration element 12050. The administration element hub 12046 may be mounted on supports. The length of the administration element 12050 may project distally into the administration element hub 12051 at the distal end 12055 in such a way that it does not project outside of the dispensing device 12000 in the inactive state.

When activated by depression of the activator 12001, the pressurized fluid source 12005 may interact with a fluid source initiator 12015, which may provide a pressurized fluid from the pressurized fluid source 12005, which may pass through the entry passage 12012 and fill the pressure chamber 12017. Pressure on the diaphragm 12040 and plunger 12020 may induce distal translation of the primary container 12025, transferring force to the piercing end 12045 of the administration element 12050, which may then puncture the septum 12037 of the primary container 12025. Continued force may separate the administration element hub 12046 from suspension support allowing the administration element hub to serve as a space between the primary container 12025 and the interior surface of the distal end 12055. In this configuration, the administration element 12050 projects through the administration element hub 12051. Pressure within the pressure chamber 12017 may force the plunger 12020-plunger rod 12022-stopper 12027 assembly into the primary container 12025, expelling the beneficial agent 12036 from within the storage volume 12035 through the fluid passageway of the administration element 12050 and out the distal port 12052 to an external environment.

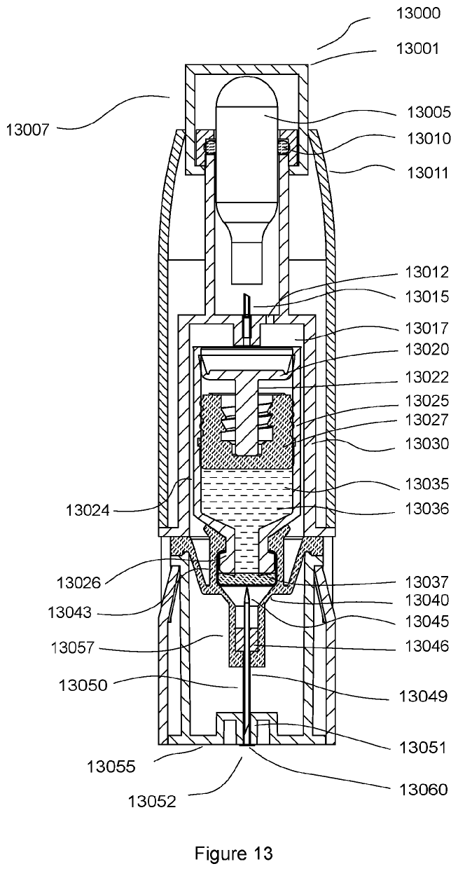

FIG. 13 illustrates a cross-sectional view of the initial pre-activation state of a dispensing device 13000 comprising an auto-injector in an inactive state. The dispensing device 13000 includes a body 13011 with an activator 13001 at the proximal end 13007. In this configuration, the auto-injector consists of three primary regions within the body 13011, also referred to herein as the housing: a region containing a pressurized fluid source 13005 of pressured gas situated distal to an activator 13001, a pressure chamber 13017 whose boundaries are defined by rigid walls that form a central barrel 13030 and a diaphragm 13040 (e.g., a flexible diaphragm) that engages and seals against the body 13011 at the distal end of the chamber. A primary container 13025 may be disposed within the central barrel, held captive at the distal end by the diaphragm 13040, which encompasses the neck of the primary container 13025 as well as the septum 13037, which seals its distal end. The proximal end of the primary container 13025 includes a plunger 13020 incorporating a plunger rod 13022 extending into an interior cavity in a stopper 13027 disposed within the primary container 13025. A beneficial agent 13036 may be disposed within the storage volume 13035 of the primary container 13025. A section of the diaphragm 13040 may extend distally to encapsulate at least part of an administration element 13050 and may further surround an administration element hub 13046 (e.g., a rigid administration element hub), forming an administration element receptacle 13057, which may comprise a diaphragm sheath. The administration element receptacle 13057 may terminate and/or seal against a sheath 13049. These may serve singly or in combination to keep the administration element 13050 sterile. The sheath 13049 may form an element of a barrier 13060 covering the distal port 13052 and/or may form an element of the diaphragm 13040. The distal end of the administration element 13050 is further supported by an administration element hub 13051, in which it may be partially disposed in the inactive state. The distal port 13052 may provide a pathway through the administration element hub 13051, and that pathway may be covered and/or sealed against an external environment by a barrier 13060, which may be attached to the sheath 13049. The barrier 13060, the sheath 13049, and/or the administration element receptacle 13057 may provide an aseptic barrier against contamination of the administration element 13050 by pathogens, pyrogens, or other environmental contaminants.

As shown in FIG. 13, the primary container 13025 may be situated within the pressure chamber 13017 and engaged with the diaphragm 13040. The diaphragm 13040 may include a receptacle 13043 with which the primary container 13025 is engaged, and the primary container 13025 may optionally include a fitment 13026 where it engages with the receptacle 13043. The primary container 13025 may include a storage volume 13035 for storing a beneficial agent 13036 and may be configured to deplete the beneficial agent 13036 through a distal port 13052 located at the distal end 13055 when a pressurized fluid is provided to the pressure chamber 13017. In one variation, the primary container 13025 may be a flexible cartridge configured to collapse in the active state when a pressurized fluid is provided into the pressure chamber 13017. In another variation, the primary container 13025 may be a rigid cartridge which can include a rigid sidewall 13024, a plunger 13020, optionally including a plunger rod 13022, and/or a stopper 13027 which may be configured to bound the storage volume 13035 against the volume of the pressure chamber 13017 and/or an interstitial volume between the plunger 13020 and stopper 13027. A rigid cartridge may include a distal end with a septum 13037 pierceable by a piercing end of an administration element 13050.

The diaphragm 13040 may include an administration element receptacle 13057, which may movably accommodate the administration element 13050, and the administration element receptacle 13057 may be located distal to the primary container 13025 at the distal end 13055 of the dispensing device 13000. A portion of the diaphragm 13040 may be adapted to deform responsive to the distal moving of the primary container 13025. The deformation may be such that the administration element 13050 and/or a piercing end 13045 thereof penetrates the primary container 13025. A portion of the diaphragm 13040 encompasses the upper portion of an administration element 13050 that consists of a piercing end 13045 and a distal port 13052 and seals to an administration element hub 13046 (e.g., a loosely affixed administration element hub) flanking the body of the administration element 13050. The administration element hub 13046 may be mounted on supports. The length of the administration element 13050 may project distally into the administration element hub 13051 at the distal end 13055 in such a way that it does not project outside of the dispensing device 13000 in the inactive state.

During activation of the dispensing device 13000, actuation of the activator 13001 may cause the pressurized fluid source 13005 to overcome a resistance to movement which may be provided by a proximal retaining element 13010, resulting in the initiation of the pressurized fluid source 13005 by the fluid source initiator 13015. Fluid released from the pressurized fluid source 13005 transfers to the pressure chamber 13017 through an entry passage 13012 disposed between the adjoining sections. Pressure within the chamber may distribute across the available surfaces, including the plunger 13020, the proximal end of the primary container 13025, and the stretchable section of the diaphragm 13040.

The dispensing device 13000 may include a barrier 13060, which may be configured to seal the distal end 13055 against an external environment in the inactive state. The barrier may cover a distal port 13052 at the distal end 13055. Such a barrier may be configured to provide an aseptic seal preventing the intrusion of pathogens, pyrogens, or other environmental contaminants into the dispensing device 13000 at the distal end 13055 and/or through the distal port 13052. The administration element 13050 may be configured to penetrate the barrier 13060 in the active state, and/or the barrier 13060 may be configured to be partially or wholly removed prior to activation of the dispensing device 13000.

In some aspects, the designs of the dispensing device disclosed herein may optionally include a body including a pressure chamber and a distal end; a primary container, including a storage volume, configured to store a beneficial agent in the inactive state. The primary container may be disposed within the pressure chamber; and a diaphragm disposed within the body, the primary container being engaged with the diaphragm, and the distal end being disposed on a distal side of the diaphragm, wherein the dispensing device is configured to transition between an inactive state and an active state when a pressurized fluid is provided to the pressure chamber by distally moving the primary container against the diaphragm causing the diaphragm to deform, including stretching of the diaphragm in a distal direction.

In some aspects, the designs of the dispensing device disclosed herein may optionally include an administration element at least partially disposed on the distal side of the diaphragm in the inactive state, wherein the administration element is configured to penetrate the primary container responsive to the distal moving of the primary container.

In some aspects, the designs of the dispensing device disclosed herein may optionally include variations wherein the administration element is configured to penetrate the primary container through the diaphragm responsive to the distal moving of the primary container.

In some aspects, the designs of the dispensing device disclosed herein may optionally include variations wherein the primary container is at least partially sealed by a septum, and wherein the administration element is configured to penetrate the primary container through the septum responsive to the distal moving of the primary container.

In some aspects, the designs of the dispensing device disclosed herein may optionally include variations with a pressurized fluid source, wherein the pressurized fluid source is adapted to provide the pressurized fluid to the pressure chamber.

In some aspects, the designs of the dispensing device disclosed herein may optionally include variations with the diaphragm comprising a receptacle engaged with the primary container, wherein when the pressurized fluid is provided to the pressure chamber the receptacle is compressed against the primary container.

In some aspects, the designs of the dispensing device disclosed herein may optionally include variations with a retaining element disposed relative to the receptacle and adapted to retain the receptacle against the primary container.

In some aspects, the designs of the dispensing device disclosed herein may optionally include variations wherein the retaining element is adapted to maintain a seal between the receptacle and the primary container.

In some aspects, the designs of the dispensing device disclosed herein may optionally include variations wherein the primary container comprises a fitment against which the receptacle compresses.

In some aspects, the designs of the dispensing device disclosed herein may optionally include variations wherein the fitment is disposed substantially within the primary container.

In some aspects, the designs of the dispensing device disclosed herein may optionally include variations wherein the distal moving of the primary container is responsive to the pressurized fluid source providing the pressurized fluid to the pressure chamber.

In some aspects, the designs of the dispensing device disclosed herein may optionally include variations wherein the primary container further includes a plunger positioned proximal to the storage volume, the plunger defining a proximal boundary of the storage volume in the inactive state.

In some aspects, the designs of the dispensing device disclosed herein may optionally include variations wherein the transition between the inactive state and the active state includes moving the plunger distally within the primary container, responsive to the penetration of the primary container by the administration element.

In some aspects, the designs of the dispensing device disclosed herein may optionally include variations wherein in the active state the plunger moves distally within the primary container to deplete the storage volume and expel the beneficial agent through the administration element.

In some aspects, the designs of the dispensing device disclosed herein may optionally include variations wherein the primary container comprises a flexible cartridge.

In some aspects, the designs of the dispensing device disclosed herein may optionally include variations wherein the transition between the active and the inactive state includes flexing the flexible cartridge responsive to penetration by the administration element.

In some aspects, the designs of the dispensing device disclosed herein may optionally include variations wherein in the active state the flexible cartridge flexes to deplete the storage volume and expel the beneficial agent through the distal end.

In some aspects, the designs of the dispensing device disclosed herein may optionally include variations wherein the administration element includes a needle, a jet injector nozzle, a cannula, a tapered fitting, or a medical device connector.

In some aspects, the designs of the dispensing device disclosed herein may optionally include variations wherein the administration element is configured to provide fluid communication between the storage volume and an external environment in the active state.

In some aspects, the designs of the dispensing device disclosed herein may optionally include variations wherein the diaphragm comprises an administration element receptacle which moveably accommodates the administration element.

In some aspects, the designs of the dispensing device disclosed herein may optionally include variations wherein a portion of the diaphragm is adapted to deform responsive to the distal moving of the primary container such that the administration element penetrates the primary container.

In some aspects, the designs of the dispensing device disclosed herein may optionally include variations with a barrier configured to aseptically seal the distal end against an external environment in the inactive state.

In some aspects, the designs of the dispensing device disclosed herein may optionally include variations wherein the administration element penetrates the barrier in the active state.

In some aspects, the designs of the dispensing device disclosed herein may optionally include variations wherein the primary container is adapted to expel the beneficial agent through the administration element when the pressure chamber is pressurized.

In some aspects, the designs of the dispensing device disclosed herein may optionally include variations wherein the diaphragm is aseptically sealed against the administration element.

In some aspects, the designs of the dispensing device disclosed herein may optionally include variations further comprising: an administration element hub, wherein the diaphragm is aseptically sealed against the distal end via the administration element hub in the inactive state.

In some aspects, the designs of the dispensing device disclosed herein may optionally include variations wherein the primary container is disposed on a proximal side of the diaphragm.

In some aspects, the designs of the dispensing device disclosed herein may optionally include variations further comprising a fluid passage providing fluid flow of a pressurized fluid from the pressure chamber when a pressurized fluid is provided to the pressure chamber.

In some aspects, the designs of the dispensing device disclosed herein may optionally include variations wherein the fluid passage provides fluid flow through the diaphragm.

In some aspects, the designs of the dispensing device disclosed herein may optionally include variations wherein the primary container retracts proximally when the pressurized fluid is expelled from the pressure chamber through the fluid passage.

In some aspects, the designs of the dispensing device disclosed herein may optionally include variations wherein a dwell time in the active state is defined by a retraction force acting on the primary container and a flow rate of the fluid flow through the fluid passage.

In some aspects, the designs of the dispensing device disclosed herein may optionally include variations wherein the storage volume of the primary container is in fluid communication with an external environment during the dwell time.

In some aspects, the designs of the dispensing device disclosed herein may optionally include variations with a distal chamber disposed on the distal side of the diaphragm within the body, the distal chamber being adapted such that the fluid flow is directed into the distal chamber in the active state.

In some aspects, the designs of the dispensing device disclosed herein may optionally include variations wherein the primary container retracts proximally when the pressure of the pressurized fluid is lower than a threshold pressure.

In some aspects, the designs of the dispensing device disclosed herein may optionally include variations further comprising a biasing element, the biasing element biasing the primary container to retract proximally when the pressure of the pressurized fluid is lower than a threshold pressure.

In some aspects, the designs of the dispensing device disclosed herein may optionally include variations wherein the diaphragm is adapted to bias the primary container to retract proximally when the pressure of the pressurized fluid is lower than a threshold pressure.

In some aspects, the designs of the dispensing device disclosed herein may optionally include variations wherein the stretching causes a portion of the diaphragm to move to engage with the distal end.

In some aspects, the designs of the dispensing device disclosed herein may optionally include variations wherein a distal portion of the primary container is sealed against a sealing portion of the diaphragm in the inactive state.

In some aspects, the designs of the dispensing device disclosed herein may optionally include variations further comprising: a seal assist biasing element configured to bias the distal portion of the primary container against the sealing portion of the diaphragm.

In some aspects, the designs of the dispensing device disclosed herein may optionally include variations further comprising: a distal vent hole configured to relieve pressure in the distal end responsive to the stretching of the diaphragm.

In some aspects, the designs of the dispensing device disclosed herein may optionally include variations with the distal end further comprising a distal port.

In some aspects, the designs of the dispensing device disclosed herein may optionally include variations wherein the distal chamber is sealed and the pressure in the distal chamber increases when the device transitions from the inactive state to the active state.

In some aspects, the designs of the dispensing device disclosed herein may optionally include variations where the increased pressure in the distal chamber facilitates the retraction of the primary container.

In some aspects, the designs of the dispensing device disclosed herein may optionally include variations wherein the diaphragm restricts fluid flow through the fluid passage in the active state, and said restriction is lessened after a dwell time and/or when the primary container begins to retract.

In some aspects, the designs of the dispensing device disclosed herein may optionally include variations where the primary container comprises a rigid port at its distal end.

In some aspects, the designs of the dispensing device disclosed herein may optionally include variations wherein the administration element is movable with the primary container when the device transitions from the inactive state to the active state.

In some aspects, the designs of the dispensing device disclosed herein may optionally include variations wherein the fluid communication is terminated, i.e. the administration element is withdrawn from the primary container, when a biasing element moves the administration device out of fluid communication with the primary container.

In some aspects, the designs of the dispensing device disclosed herein may optionally include variations with a barrier which aseptically seals the dispensing device in the inactive state.

In some aspects, the designs of the dispensing device disclosed herein may optionally include variations where the barrier forms an aseptic seal with the diaphragm.

In some aspects, the designs of the dispensing device disclosed herein may optionally include variations where the barrier includes a sheath.

In some aspects, the designs of the dispensing device disclosed herein may optionally include variations wherein the passageway is adjusted by at least one of the pressure in the chamber and the stretching of the diaphragm.

In some aspects, the designs of the dispensing device disclosed herein may optionally include variations wherein the administration element is fixed to a distal wall.

In some aspects, the designs of the dispensing device disclosed herein may optionally include variations wherein the dwell time ends when an administration element is dislodged from the primary container, ending fluid communication between the storage volume and an external environment.

While the technology has been described in language that is specific to certain structures and materials, it is to be understood that the invention defined in the appended claims is not necessarily limited to the specific structures and materials described. Rather, the specific aspects are described as forms of implementing the claimed invention. Specific features of the technology should generally be regarded as optional and combinable with respect to each other and with respect any design as a whole, except where otherwise explicitly noted. Because many embodiments of the invention can be practiced without departing from the spirit and scope of the invention, the invention resides solely in the claims.

1. A dispensing device for dispensing a beneficial agent configured to transition between an inactive state and an active state comprising:
a body including a pressure chamber and a distal end;
a primary container a including a storage volume configured to store a beneficial agent in the inactive state, the primary container disposed within the pressure chamber; and
a diaphragm disposed within the body, the primary container being engaged with the diaphragm, and the distal end being disposed on a distal side of the diaphragm, wherein the dispensing device is configured to transition between an inactive state and an active state when a pressurized fluid is provided to the pressure chamber by distally moving the primary container against the diaphragm causing the diaphragm to deform, including stretching of the diaphragm in a distal direction.

2. The dispensing device as claimed in claim 1, the distal end further comprising:
an administration element at least partially disposed on the distal side of the diaphragm in the inactive state, wherein the administration element is configured to penetrate the primary container responsive to the distal moving of the primary container.

3. The dispensing device as claimed in claim 2, wherein the administration element is configured to penetrate the primary container through the diaphragm responsive to the distal moving of the primary container.

4. The dispensing device as claimed in claim 2, the primary container at least partially sealed by a septum, wherein the administration element is configured to penetrate the primary container through the septum responsive to the distal moving of the primary container.

5. The dispensing device as claimed in claim 2, further comprising:
a pressurized fluid source, wherein the pressurized fluid source is adapted to provide the pressurized fluid to the pressure chamber.

6. The dispensing device as claimed in claim 5, further comprising:
the diaphragm comprising a receptacle engaged with the primary container, wherein when the pressurized fluid is provided to the pressure chamber the receptacle is compressed against the primary container.

7. The dispensing device as claimed in claim 6, further comprising a retaining element disposed relative to the receptacle and adapted to retain the receptacle against the primary container.

8. The dispensing device as claimed in claim 7, wherein the retaining element is adapted to maintain a seal between the receptacle and the primary container.

9. The dispensing device as claimed in claim 6, wherein the primary container comprises a fitment against which the receptacle compresses.

10. The dispensing device as claimed in claim 9, wherein the fitment is disposed substantially within the primary container.

11. The dispensing device as claimed in claim 5, wherein the distal moving of the primary container is responsive to the pressurized fluid source providing the pressurized fluid to the pressure chamber.

12. The dispensing device as claimed in claim 11, wherein the primary container further includes a plunger positioned proximal to the storage volume, the plunger defining a proximal boundary of the storage volume in the inactive state.

13. The dispensing device as claimed in claim 12, wherein the transition between the inactive state and the active state includes moving the plunger distally within the primary container, responsive to the penetration of the primary container by the administration element.

14. The dispensing device as claimed in claim 12, wherein in the active state the plunger moves distally within the primary container to deplete the storage volume and expel the beneficial agent through the administration element.

15. The dispensing device as claimed in claim 11, wherein the primary container comprises a flexible cartridge.

16. The dispensing device as claimed in claim 15, wherein the transition between the active state and the inactive state includes flexing the flexible cartridge responsive to penetration by the administration element.

17. The dispensing device of claim 15, wherein in the active state the flexible cartridge flexes to deplete the storage volume and expel the beneficial agent through the distal end.

18. The dispensing device of claim 2, wherein the administration element includes a needle, a jet injector nozzle, a cannula, a tapered fitting, or a medical device connector.

19. The dispensing device as claimed in claim 2, wherein the administration element is configured to provide fluid communication between the storage volume and an external environment in the active state.

20. The dispensing device as claimed in claim 2, wherein the diaphragm comprises an administration element receptacle that movably accommodates the administration element.

21-47. (canceled)
